(19) United States
(12) Patent Application Publication
(10) Pub. No.: US 2022/0096624 A1
(43) Pub. Date: Mar. 31, 2022

(54) EXTRACELLULAR VESICLES HARBORING A SPIKE PROTEIN, NUCLEIC ACIDS FOR PRODUCING THE SAME, AND METHOD OF IMMUNIZING A SUBJECT AGAINST SARS-CoV-2 USING THE SAME

(71) Applicant: CILOA, Montpellier (FR)

(72) Inventors: Bernadette Nadine TRENTIN, Saint-André-de-Sangonis (FR); Katarzyna POLAK, Montpellier (FR); Zaïne El Abiddine Robert MAMOUN, Saint-André-de-Sangonis (FR)

(73) Assignee: CILOA, Montpellier (FR)

(21) Appl. No.: 17/036,569

(22) Filed: Sep. 29, 2020

(51) Int. Cl. A61K 39/215; C07K 14/005; A61K 39/39; A61P 31/14; A61K 47/69; A61K 47/64

(52) U.S. Cl. CPC A61K 39/215; C07K 14/005; A61K 39/39; A61K 2039/545; A61K 47/6901; A61K 47/646; A61P 31/14

(57) ABSTRACT

A nucleic acid including (i) a sequence of a S gene coding for a Spike protein and (ii) a sequence coding for a pilot peptide which interacts with ESCRT proteins. Also, extracellular vesicles, in particular exosomes, harboring at their external surface a Spike protein, obtainable by transfecting cells with the nucleic acid. Further, a method of immunizing a subject against a virus of the Orthocoronavirinae subfamily, in particular against SARS-CoV-2, by DNA prime-protein boost using the nucleic acid and the extracellular vesicles of the invention.

### FIELD

The present invention relates to a nucleic acid comprising (i) a sequence of a S gene coding for a Spike protein and (ii) a sequence coding for a pilot peptide which interacts with ESCRT proteins. It further relates to extracellular vesicles, in particular exosomes, harboring at their external surface a Spike protein, obtainable by transfecting cells with the nucleic acid. It further relates to a method of immunizing a subject against a virus of the Orthocoronavirinae subfamily, in particular against SARS-CoV-2, by DNA prime-protein boost using the nucleic acid and the extracellular vesicles of the invention.

### BACKGROUND

Unique efforts to develop a vaccine against the Severe Acute Respiratory Syndrome Coronavirus-2 (SARS-Cov-2) virus correspond to the level of the worldwide threat. Most of the vaccine candidates are based on the Spike (S) protein of the virus. In the absence of sufficient information on coronaviruses, it is important to learn on the lessons of the past on other enveloped viruses.

Coronaviruses, human immunodeficiency viruses (HIV), influenza viruses and others, possess type I transmembrane envelope proteins which enable their cellular entry. These proteins are synthesized as trimers that are then cleaved in two mature proteins S1 and S2 that form a “head” and a “stem” region, the later anchoring the mature trimer into the viral membrane and being responsible for the virus-cell membrane fusion. Proteins S1 and S2 are the equivalents of proteins SU and TM in HIV and of proteins HA1 and HA2 in influenza viruses.

The immunogenicity of viral envelope proteins is decreased by surface glycosylation and by the trimeric structure of the Spike, that occlude important epitopes. Decades on HIV vaccine development revealed the absolute necessity of using a fully native envelope protein embedded in a membrane in order to obtain a high-quality immune response. Surprisingly, protein SU-based vaccines provide no protection from HIV infection in human, although they could trigger neutralizing antibody production in small animals and primates (Mascola et al., 1996. J Infect Dis. 173(2):340-348). Additionally, influenza virus-neutralizing antibodies targeting protein HA1 epitopes are usually susceptible to strain variability (Hensley et al., 2009. Science. 326(5953):734-736).

The stem domain (protein TM in HIV, HA2 in influenza viruses or S2 in coronaviruses) is more conserved than the head domain (protein SU in HIV, HA1 in influenza viruses or S1 in coronaviruses). In HIV, the stem contains a highly conserved Membrane Proximal External Region (MPER) potent neutralizing epitope (Zwick, 2005. AIDS. 19(16):1725-1737). At the same time, influenza viruses stem is the target of broadly neutralizing antibodies (Wang et al., 2018. J Virol. 92(12):e00247-18). Thus, vaccines that mount broad protection against either HIV, influenza viruses, or other enveloped viruses, must elicit anti-stem domain antibodies (Khanna et al., 2014. Biomed Res Int. 2014:546274). Consequently, chimeric viruses or virus-like particles (VLPs) harboring native envelope proteins seem the best platforms as a result of taking the conformation of potential MPER peptides and the scaffold feature of lipids into consideration (Liu et al., 2018. Protein Cell. 9(7):596-615).

In addition to neutralizing antibodies, a consensus arises that triggering a strong T-cell immune response, even in the absence of cross-reactive neutralizing antibodies, correlates with a protection against several viral strains and for a longer time (Sridhar et al., 2013. Nat Med. 19(10):1305-1312; Wilkinson et al., 2012. Nat Med. 18(2):274-280). In addition, work on MERS-CoV and SARS-CoV revealed the importance of both humoral and cellular immune responses to induce a protection (Mubarak et al., 2019. J Immunol Res. 2019:6491738; Xu & Gao, 2004. Cell Mol Immunol. 1(2):119-122). Finally, there is mounting evidence for a positive correlation between the resistance to the virus, the level of T cell response in SARS-Cov-2 infected people and T-cell cross reactivity against the S2 subunit (Braun et al., 2020. Nature. 2020; Sekine et al., 2020. Cell. 2020).

Among common immunization strategies is the so-called “DNA prime-protein boost” strategy, which comprises administering a DNA vaccine encoding the antigen in vivo during the priming step, then the antigen in proteinic form, produced in vitro, during the boosting step (Barnett et al., 1997. Vaccine. 15(8):869-873; Pal et al., 2006. Virology. 348(2):341-353; Wang et al., 2008. Vaccine. 26(31):3947-3957; Richmond et al., 1998. J Virol. 72(11):9092-9100; Liu et al., 2020. Sci Rep. 10(1):4144; Wang et al., 2004. Vaccine. 22(27-28):3622-3627; Xiao-wen et al., 2005. Vaccine. 23(14):1649-1656). In the field of coronaviruses, International patent application WO2016138160 has taught a method for inducing an immune response to the MERS-CoV Spike protein in the form of DNA prime-protein boost, with a priming step comprising administering a nucleic acid molecule encoding the MERS-CoV Spike protein and a boosting step comprising administering the MERS-CoV S1 subunit.

However, there remains a need for improved immunization strategies, eliciting both neutralizing antibodies and a strong T-cell immune response.

Here, the Inventors offer a novel immunization strategy, more closely reflecting the three-dimensional conformation of the antigen and its structural arrangement vis-a-vis the lipid membrane in which it is anchored.

Extracellular vesicles (EVs; of which, exosomes) are nano-sized particles secreted by all eukaryotic cells, capable of carrying antigenic information and triggering both humoral and cellular immune responses. To exploit EVs as natural vaccines, the Inventors have developed a technology that allows the natural loading of EVs with any fully native membrane protein (granted patents EP 2 268 816 and U.S. Pat. No. 9,546,371; Desplantes et al., 2017. Sci Rep. 7(1):1032). Briefly, a protein of interest is fused to a proprietary Pilot Peptide (CilPP), which interacts with the Endosomal Sorting Complexes Required for Transport (ESCRT) cellular machinery to sort the resulting chimeric protein onto EVs.

By taking advantage of this technology, the Inventors present herein a SARS-CoV-2 vaccine, CoVEVax, which displays S1/S2 subunits naturally embedded in the membrane of EVs (hereafter, S-EVs), perfectly mimicking the original virus with its full transmembrane and membrane proximal external region (MPER) subdomains.

### SUMMARY

The present invention relates to a nucleic acid comprising:

(i) a sequence of a S gene coding for a Spike protein from a virus of the Orthocoronavirinae subfamily, or a variant thereof; and
(ii) a sequence coding for a pilot peptide which interacts with ESCRT proteins.

In one embodiment, the virus of the Orthocoronavirinae subfamily is Severe Acute Respiratory Syndrome Coronavirus 2 (SARS-CoV-2).

In one embodiment, the S gene has a nucleic acid sequence coding for a Spike protein with SEQ ID NO: 2, or a variant thereof.

In one embodiment, the S gene has a nucleic acid sequence coding for a variant of the Spike protein with SEQ ID NO: 6.

In one embodiment, the pilot peptide comprises at least one YxxL motif with SEQ ID NO: 17 or DYxxL motif with SEQ ID NO: 20, and at least one PxxP motif with SEQ ID NO: 24, in which “x” represents any amino acid residue.

In one embodiment, the pilot peptide comprises an amino acid sequence with SEQ ID NO: 8 or a variant thereof, with the proviso that a variant of SEQ ID NO: 8 retains three YxxL motifs with SEQ ID NO: 17 and four PxxP motifs with SEQ ID NO: 24, in which “x” represents any amino acid residue.

In one embodiment, the nucleic acid is inserted into a nucleic acid expression vector, and is operably linked to regulatory elements.

The present invention also relates to an extracellular vesicle harboring at its external surface a Spike protein from a virus of the Orthocoronavirinae subfamily “S-EV”, or a variant thereof.

In one embodiment, the virus of the Orthocoronavirinae subfamily is Severe Acute Respiratory Syndrome Coronavirus 2 (SARS-CoV-2) In one embodiment, the extracellular vesicle is an exosome.

In one embodiment, the extracellular vesicle is an exosome having a diameter ranging from about 30 nm to about 120 nm.

In one embodiment, the extracellular vesicle is obtainable by a method comprising steps of:

1) transfecting cells with a nucleic acid comprising:
a. a sequence of a S gene coding for a Spike protein from a virus of the Orthocoronavirinae subfamily, or a variant thereof; and
b. a sequence coding for a pilot peptide which interacts with ESCRT proteins;
2) culturing the transfected cells for a time sufficient to allow extracellular vesicle production; and
3) purifying said extracellular vesicle.

The present invention also relates to a population of extracellular vesicles according to the invention.

The present invention also relates to a method of immunizing a subject in need thereof against a virus of the Orthocoronavirinae subfamily, comprising the steps of:

1) at least one priming step, comprising administering to said subject a nucleic acid comprising:
a. a sequence of a S gene coding for a Spike protein from a virus of the Orthocoronavirinae subfamily, or a variant thereof; and
b. a sequence coding for a pilot peptide which interacts with ESCRT proteins; and
2) at least one boosting step, comprising:
a. administering to said subject an extracellular vesicle harboring at its external surface a Spike protein from a virus of the Orthocoronavirinae subfamily “S-EV”, or a variant thereof, or
b. administering to said subject a trimeric Spike protein from a virus of the Orthocoronavirinae subfamily, thereby immunizing the subject against a virus of the Orthocoronavirinae subfamily.

In one embodiment, the virus of the Orthocoronavirinae subfamily is Severe Acute Respiratory Syndrome Coronavirus 2 (SARS-CoV-2).

In one embodiment, the period of time between the at least one priming step and the at least one boosting step ranges from about 2 weeks to about 1 month.

In one embodiment, the method comprises two iterations of the priming step and one iteration of the boosting step.

In one embodiment, the period of time between each iteration ranges from about 2 weeks to about 1 month.

### BRIEF DESCRIPTION OF THE DRAWINGS

FIGS. 1A and 1B show the predicted B- and T-cell epitope homology within the Spike protein of SARS-CoV-2, SARS-CoV, MERS, common cold viruses, HIV gp41 and CoVEVax vaccine candidates.

1A is an alignment of HIV gp41 MPER-containing sequences. Alignment between HIV strains shows conservation of tryptophan rich region (in bold) and cholesterol interaction domain within predicted neutralizing epitopes.

1B shows an alignment of Spike proteins between different coronaviruses [CoV-229E, CoV-NL63, CoV-HKU1, CoV-OC43, MERS, SARS-CoV and SARS-CoV-2], and the constructs used in this study [S-EV and S-Trim]. Tryptophan rich regions and T cell epitopes present within the corresponding Membrane Proximal External Region [MPER] and transmembrane domain [TM] are retained in the Spike protein presented by S-EVs but absent in the S-Trim truncated at residue 1219 (SEQ ID NO: 6 numbering; or residue 1208 with SEQ ID NO: 2 numbering) and fused to a Foldon trimerization motif. S-EV and S-Trim comprise a modified signal peptide [SP]. S-EV comprises a proprietary pilot peptide (CilPP). Proline mutations were introduced in indicated locations (SEQ ID NO: 2 numbering).

FIGS. 2A to 2H show the production and characterization of the CoVEVax vaccine candidates.

2A is a scheme of the production of EV-based vaccine candidates. Three different immunogens were generated to mount immune response against SARS-CoV-2 in mice. DNA^{S-EV} vector allows in situ production of autologous S-EVs upon injection to mice. S-EVs and S-Trim are produced in mammalian cells (HEK293T) in vitro.

2B is a set of two gels showing Western blot analysis of cellular extracts from HEK293T cells transfected with DNA^{S-Trim} and from purified S-Trim (left panel: revealed with anti-S1 antibodies; right panel: revealed with anti-S2 antibodies). The arrow labelled S indicate the size expected for a non-mature Spike protein.

2C is a gel showing Western blot analysis of cellular extracts from HEK293T cells transfected with DNA^{S-EV} and revealed with anti-CilPP antibodies. The arrow labelled S indicate the size expected for a non-mature Spike protein; the arrow labelled S2 indicate the size expected for a mature S2 subunit.

2D is a set of three graphs showing the purified EVs characterization by ExoView platform. EVs expressing Spike protein are captured with either CD81, CD63 or CD9.

2E is a set of two graphs showing the ELISA analysis of proteins S1 and S2 displayed on S-EVs. Results are shown as OD_{450 nm} values.

2F is a set of two gels showing Western blot analysis of Spike protein expression on S-EVs. The arrow labelled S indicate the size expected for a non-mature Spike protein; the arrow labelled S1 indicate the size expected for a mature S1 subunit; the arrow labelled S2 indicate the size expected for a mature S2 subunit.

2G is a gel showing Western blot analysis of S-EVs purified from HEK293T cells transfected with DNA^{S-EV} and revealed with anti-CilPP antibodies in reducing conditions. The arrow labelled S indicate the size expected for a non-mature Spike protein; the arrow labelled S2 indicate the size expected for a mature S2 subunit.

2H is a gel showing Western blot analysis of S-EVs purified from HEK293T cells transfected with DNA^{S-EV} and revealed with anti-CilPP antibodies in non-reducing conditions. The arrow labelled S indicate the size expected for a non-mature and trimeric Spike protein.

FIGS. 3A to 3D shows the humoral response in mice immunized with CoVEVax.

3A is a schema showing the CoVEVax immunization study design. Four groups of female BALB/cAnNCrl mice (n=6) received two prime injections at day 0 and 21 and one boost injection at day 42. Mice were bled at day 0, 21, 42 and 63 and spleens were harvested at day 63 to analyze antigen specific cellular response.

3B is a set of two graphs showing the specific IgG response to S1 (left panel) or S2 (right panel) of 2 overlapping peptide pools (each representing respectively, whole S1 or whole S2) at day 63 measured by ELISA for each of the 4 groups. Results are presented as reciprocal endpoint dilution titers. Data points present mean±SEM. Dashed line indicates mean titers. ns: non-significant; **: p<0.01 calculated by Mann-Whitney test.

3C is a set of two graphs showing the specific IgG response to S1 (left panel) and S2 (right panel) peptide pools at day 42 measured by ELISA for each of the 4 groups. Results are presented as reciprocal endpoint dilution titers.

3D is a set of two graphs showing the pseudovirus neutralizing antibody titers with sera collected at day 63. Titers correspond to the sera dilutions neutralizing 50% of the virus. Data points present mean±SEM. Dashed line indicates mean titers. ns: non-significant; **: p<0.01 calculated by Mann-Whitney test.

FIG. 4 shows the cellular immune response in mice immunized with CoVEVax. The set of two graphs shows the IFNγ ELISpot analysis of antigen specific cellular response. Total T-cells were isolated from pooled splenocytes (from 3 mice) and stimulated with either S1 (left panel) or S2 (right panel) SARS-CoV-2 peptide pools. Results are presented as a number of spot forming cells (SPC) per 1×10⁶ T-cells.

### DETAILED DESCRIPTION

In the present invention, the following terms have the following meanings.

“Adjuvant” refers to a molecule or complex of molecules which allow(s) or otherwise facilitate(s) (1) the mobilization of antigen-presenting and/or polymorphonuclear cells; (2) the antigen uptake and presentation of the antigen(s) in a vaccine by antigen-presenting cells; (3) the secretion of proteins by antigen-presenting cells; (4) the recruitment, targeting and activation of antigen-specific cells; (5) the modulation of activities that regulate the ensuing immune responses; and/or (6) the protection of the antigen from degradation and elimination.

“ESCRT” or “endosomal sorting complexes required for transport” refers originally to a cellular machinery made up of five multi-subunit protein complexes, which act cooperatively at specialized endosomes to facilitate the movement of specific cargoes from the limiting membrane into vesicles that bud into the endosome lumen. This machinery is hijacked by several envelope viruses to bud from cellular membranes, including the plasma membrane.

“Exosome” refers to an extracellular vesicle that is produced in the endosomal compartment of eukaryotic cells (Théry et al., 2018. J Extracell Vesicles. 7(1):1535750; Yáñlez-Mó et al., 2015. J Extracell Vesicles. 4:27066; van Niel et al., 2018. Nat Rev Mol Cell Biol. 19(4):213-228). Typically, exosomes harbor at their surface the CD81, CD63 and CD9 markers.

“Expression vector” refers to a vector capable of directing expression of a nucleic acid sequence of interest (such as, e.g., a nucleic acid according to the present invention) in an appropriate host cell, comprising a promoter operatively linked to the nucleic acid sequence of interest, itself operatively linked to a termination sequence.

“Extracellular vesicle” refers to any vesicle composed of a lipid bilayer that are naturally released from a cell and comprise a cytosolic fraction of said cell. This expression in particular includes vesicles secreted into the extracellular space, i.e., “exosomes”.

“Isolate” refers to a genetic variant of a given viral strain. In brief, after rounds of replication, a virus-infected cell will contain a population of viral genomes, and virions derived from these viral genomes will vary slightly from each other. Likewise, a sample taken from a virus culture or an infected animal will contain numerous virions, many of which vary slightly. Consequently, an “isolate” refers to populations of slightly different viruses from a same strain, collected from a single source (a single subject, a single sample, etc.). By extension, the sequence of an “isolate” is thus a consensus sequence of the population of viral genomes collected from a single source. In the frame of this disclosure, SARS-CoV-2 isolate “Wuhan-Hu-1” with GenBank accession number MN908947.3, version 3 of Mar. 18, 2020, is considered as the reference strain for all SARS-CoV-2 viruses. However, to date, over 100 000 viral genomic sequences of different SARS-CoV-2 isolates have been submitted on the GISAID EpiCoV™ database.

“Isolated” and any declensions thereof, as well as “purified” and any declensions thereof, are used interchangeably, and mean that a molecular entity to which it refers (e.g., a protein or peptide, a nucleic acid, an extracellular vesicle, etc.) is substantially free of other components (i.e., of contaminants) found in the natural environment in which said molecular entity is normally found. Preferably, an isolated or purified molecular entity (e.g., an isolated or purified protein or peptide, an isolated or purified nucleic acid, an isolated or purified extracellular vesicle, etc.) is substantially free of other molecular entities with which it is associated in a cell or a virus. By “substantially free”, it is meant that said isolated or purified molecular entity represents more than 50% of a heterogeneous composition (i.e., is at least 50% pure), preferably, more than 60%, more than 70%, more than 80%, more than 90%, more than 95%, and more preferably still more than 98% or 99%. Purity can be evaluated by various methods known by the one skilled in the art, including, but not limited to, chromatography, gel electrophoresis, immunoassay, composition analysis, biological assay, and the like.

“Kit-of-parts” refers to a kit comprising a plurality of (different) items that may be functionally used together, concomitantly or one after another.

“Regulatory element” refers to nucleic acid sequences which are necessary or desirable to affect the expression of coding and non-coding sequences of interest (such as, e.g., a nucleic acid according to the present invention), to which they are operatively linked. Examples of regulatory elements include, but are not limited to, initiation signals, enhancers, regulators, promoters, and termination sequences. The nature and use of such regulatory sequences can differ, as is well known to the one skilled in the art, depending upon the host organism or cell.

“S gene” refers to a gene found in viruses of the Orthocoronavirinae subfamily, coding for the Spike protein.

“SARS-CoV-2”, also known as “severe acute respiratory syndrome coronavirus 2”, “2019 novel coronavirus”, “2019-nCoV”, “HCoV-19” or “hCoV-19”, is a strain of betacoronavirus that causes coronavirus disease 2019 (also known as COVID-19). SARS-CoV-2 complete reference genome is available online, under GenBank accession number MN908947.3, version 3 of Mar. 18, 2020. It comprises 10 genes: orflab, S, orf3a, E, M, orf6, orf7a, orf8, N, and orf10.

“Spike” or “Spike protein”, also known as “surface glycoprotein”, refers to a transmembrane protein, also termed peplomer, found in viruses of the Orthocoronavirinae subfamily, and protruding from the virus surface as spikes, hence its name. In SARS-CoV-1 and SARS-CoV-2, it allows the virus to bind to its host cell via the cell's receptor angiotensin-converting enzyme 2 (ACE2), and uses the serine protease TMPRSS2 to initiate entry and infection (Hoffmann et al., 2020. Cell. 181(2):271-280.e8). In vivo, the Spike protein is found on viruses as homotrimers, each consisting of the two subunits: a “head” called S1 and a “stem” called S2 (Wrapp et al., 2020. Science. 367(6483):1260-1263; Walls et al., 2020. Cell. 181(2):281-292.e6; Yuan et al., 2020. Science. 368(6491):630-633). The amino acid sequence of the Spike protein of the reference strain of SARS-CoV-2 (Wuhan-Hu-1 isolate, complete genome available under GenBank accession number MN908947.3, version 3 of Mar. 18, 2020) is as set forth in SEQ ID NO: 2. It is composed of an extracellular domain (amino acid residues 13 to 1217 of SEQ ID NO: 2), a transmembrane domain (amino acid residues 1218 to 1234 of SEQ ID NO: 2) and a cytoplasmic domain (amino acid residues 1235 to 1273 of SEQ ID NO: 2). Subunit S1 corresponds to amino acid residues 13 to 685 of SEQ ID NO: 2 and subunit S2 corresponds to amino acid residues 686 to 1273 of SEQ ID NO: 2. In the present disclosure, it is readily understood that the Spike protein with SEQ ID NO: 2 is only a representative protein; hence, the expression “Spike protein of SARS-CoV-2” broadly encompasses Spike proteins of any isolate of the SARS-CoV-2 virus. One skilled in the art can easily determine amino acid residue correspondence between the representative protein sequence and any sequence of a Spike protein from another isolate of the SARS-CoV-2 virus.

“Trimerization domain” refers to an amino acid sequence within a polypeptide or protein that promotes self-assembly by associating with two other trimerization domains to form a trimer. Typically, trimerization domains comprise an amino acid sequence able to form an α-helical coiled-coil domain or an isoleucine zipper domain.

“Vector” refers to a nucleic acid capable of transporting a nucleic acid of interest (such as, e.g., a nucleic acid according to the present invention) to which it has been linked. Vectors capable of directing the expression of a nucleic acid of interest (such as, e.g., a nucleic acid according to the present invention) are referred to as “expression vectors”. In general, expression vectors are in the form of plasmids. Herein, the terms “plasmid” and “vector” are used interchangeably. However, other forms of expression vectors, which serve equivalent functions, are also encompassed under the term vector.

An object of the present invention is a nucleic acid “DNA^{S-EV}” Comprising:

(i) a sequence of a S gene coding for a Spike protein from a virus of the Orthocoronavirinae subfamily, or a variant thereof; and
(ii) a sequence coding for a pilot peptide which interacts with ESCRT proteins.

Orthocoronavirinae, more commonly known as coronaviruses, are enveloped, positive-sense single-stranded RNA viruses which infect birds and mammals. Coronaviruses are divided into four genera: alphacoronavirus, betacoronavirus, gammacoronavirus and deltacoronavirus. Alphacoronaviruses and betacoronaviruses infect mammals, while gammacoronaviruses and deltacoronaviruses primarily infect birds.

Exemplary species of alphacoronaviruses include, but are not limited to, alphacoronavirus 1 (also known as transmissible gastroenteritis virus, transmissible gastroenteritis coronavirus, or TGEV), human coronavirus 229E (also known as HCoV-229E), human coronavirus NL63 (also known as HCoV-NL63), miniopterus bat coronavirus 1 (also known as Bat-CoV MOP1), miniopterus bat coronavirus HKU8 (also known as Bat-CoV HKU8), porcine epidemic diarrhea virus (also known as PED virus or PEDV), rhinolophus bat coronavirus HKU2 (also known as Chinese horseshoe bat virus or Bat-CoV HKU2), and scotophilus bat coronavirus 512 (also known as Bat-CoV 512).

Exemplary species of betacoronaviruses include, but are not limited to, betacoronavirus 1 (including, without limitation, bovine coronavirus and human coronavirus OC43), hedgehog coronavirus 1, human coronavirus HKU1 (also known as HCoV-HKU1), Middle East respiratory syndrome-related coronavirus (also known as MERS-CoV, EMC/2012 or HCoV-EMC/2012), murine coronavirus (also known as M-CoV), pipistrellus bat coronavirus HKU5 (also known as Bat-CoV HKU5), rousettus bat coronavirus HKU9 (also known as HKU9-1), severe acute respiratory syndrome-related coronavirus (also known as SARSr-CoV or SARS-CoV; and including, without limitation, SARS-CoV and SARS-CoV-2), and tylonycteris bat coronavirus HKU4 (also known as Bat-CoV HKU4).

Exemplary species of gammacoronaviruses include, but are not limited to, avian coronavirus (also known as IBV) and beluga whale coronavirus SW1 (also known as Whale-CoV SW1).

Exemplary species of deltacoronaviruses include, but are not limited to, bulbul coronavirus HKU11 (also known as Bulbul-CoV HKU11) and porcine coronavirus HKU15 (also known as porcine coronavirus HKU15 or PorCoV HKU15).

In one embodiment, the virus of the Orthocoronavirinae subfamily is a virus of the betacoronavirus genus.

In one embodiment, the virus of the Orthocoronavirinae subfamily is a virus of the betacoronavirus genus, and severe acute respiratory syndrome-related coronavirus species.

In one embodiment, the virus of the Orthocoronavirinae subfamily is a virus of the betacoronavirus genus, severe acute respiratory syndrome-related coronavirus species and strain SARS-CoV-2.

In one embodiment, the virus of the Orthocoronavirinae subfamily is SARS-CoV-2.

In one embodiment, the S gene of SARS-CoV-2 has a nucleic acid sequence coding for a Spike protein with SEQ ID NO: 2, or a variant thereof. In one embodiment, the S gene coding for a Spike protein of SARS-CoV-2 with SEQ ID NO: 2 or a variant thereof has a nucleic acid sequence with SEQ ID NO: 1, or a variant thereof.

-S gene from SARS-CoV-2 Wuhan-Hu-1 isolate (MN908947.3)
SEQ ID NO: 1
ATGTTTGTTTTTCTTGTTTTATTGCCACTAGTCTCTAGTCAGTGTGTTAATCTTACA
ACCAGAACTCAATTACCCCCTGCATACACTAATTCTTTCACACGTGGTGTTTATTA
CCCTGACAAAGTTTTCAGATCCTCAGTTTTACATTCAACTCAGGACTTGTTCTTAC
CTTTCTTTTCCAATGTTACTTGGTTCCATGCTATACATGTCTCTGGGACCAATGGT
ACTAAGAGGTTTGATAACCCTGTCCTACCATTTAATGATGGTGTTTATTTTGCTTC
CACTGAGAAGTCTAACATAATAAGAGGCTGGATTTTTGGTACTACTTTAGATTCG
AAGACCCAGTCCCTACTTATTGTTAATAACGCTACTAATGTTGTTATTAAAGTCTG
TGAATTTCAATTTTGTAATGATCCATTTTTGGGTGTTTATTACCACAAAAACAACA
AAAGTTGGATGGAAAGTGAGTTCAGAGTTTATTCTAGTGCGAATAATTGCACTTT
TGAATATGTCTCTCAGCCTTTTCTTATGGACCTTGAAGGAAAACAGGGTAATTTC
AAAAATCTTAGGGAATTTGTGTTTAAGAATATTGATGGTTATTTTAAAATATATT
CTAAGCACACGCCTATTAATTTAGTGCGTGATCTCCCTCAGGGTTTTTCGGCTTTA
GAACCATTGGTAGATTTGCCAATAGGTATTAACATCACTAGGTTTCAAACTTTAC
TTGCTTTACATAGAAGTTATTTGACTCCTGGTGATTCTTCTTCAGGTTGGACAGCT
GGTGCTGCAGCTTATTATGTGGGTTATCTTCAACCTAGGACTTTTCTATTAAAATA
TAATGAAAATGGAACCATTACAGATGCTGTAGACTGTGCACTTGACCCTCTCTCA
GAAACAAAGTGTACGTTGAAATCCTTCACTGTAGAAAAAGGAATCTATCAAACT
TCTAACTTTAGAGTCCAACCAACAGAATCTATTGTTAGATTTCCTAATATTACAA
ACTTGTGCCCTTTTGGTGAAGTTTTTAACGCCACCAGATTTGCATCTGTTTATGCT
TGGAACAGGAAGAGAATCAGCAACTGTGTTGCTGATTATTCTGTCCTATATAATT
CCGCATCATTTTCCACTTTTAAGTGTTATGGAGTGTCTCCTACTAAATTAAATGAT
CTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTAC
CAGATGATTTTACAGGCTGCGTTATAGCTTGGAATTCTAACAATCTTGATTCTAA
GGTTGGTGGTAATTATAATTACCTGTATAGATTGTTTAGGAAGTCTAATCTCAAA
CCTTTTGAGAGAGATATTTCAACTGAAATCTATCAGGCCGGTAGCACACCTTGTA
ATGGTGTTGAAGGTTTTAATTGTTACTTTCCTTTACAATCATATGGTTTCCAACCC
ACTAATGGTGTTGGTTACCAACCATACAGAGTAGTAGTACTTTCTTTTGAACTTCT
ACATGCACCAGCAACTGTTTGTGGACCTAAAAAGTCTACTAATTTGGTTAAAAAC
AAATGTGTCAATTTCAACTTCAATGGTTTAACAGGCACAGGTGTTCTTACTGAGT
CTAACAAAAAGTTTCTGCCTTTCCAACAATTTGGCAGAGACATTGCTGACACTAC
TGATGCTGTCCGTGATCCACAGACACTTGAGATTCTTGACATTACACCATGTTCTT
TTGGTGGTGTCAGTGTTATAACACCAGGAACAAATACTTCTAACCAGGTTGCTGT
TCTTTATCAGGATGTTAACTGCACAGAAGTCCCTGTTGCTATTCATGCAGATCAA
CTTACTCCTACTTGGCGTGTTTATTCTACAGGTTCTAATGTTTTTCAAACACGTGC
AGGCTGTTTAATAGGGGCTGAACATGTCAACAACTCATATGAGTGTGACATACCC
ATTGGTGCAGGTATATGCGCTAGTTATCAGACTCAGACTAATTCTCCTCGGCGGG
CACGTAGTGTAGCTAGTCAATCCATCATTGCCTACACTATGTCACTTGGTGCAGA
AAATTCAGTTGCTTACTCTAATAACTCTATTGCCATACCCACAAATTTTACTATTA
GTGTTACCACAGAAATTCTACCAGTGTCTATGACCAAGACATCAGTAGATTGTAC
AATGTACATTTGTGGTGATTCAACTGAATGCAGCAATCTTTTGTTGCAATATGGC
AGTTTTTGTACACAATTAAACCGTGCTTTAACTGGAATAGCTGTTGAACAAGACA
AAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCA
AGCAAGAGGTCATTTATTGAAGATCTACTTTTCAACAAAGTGACACTTGCAGATG
CTGGCTTCATCAAACAATATGGTGATTGCCTTGGTGATATTGCTGCTAGAGACCT
CATTTGTGCACAAAAGTTTAACGGCCTTACTGTTTTGCCACCTTTGCTCACAGATG
AAATGATTGCTCAATACACTTCTGCACTGTTAGCGGGTACAATCACTTCTGGTTG
GACCTTTGGTGCAGGTGCTGCATTACAAATACCATTTGCTATGCAAATGGCTTAT
AGGTTTAATGGTATTGGAGTTACACAGAATGTTCTCTATGAGAACCAAAAATTGA
TTGCCAACCAATTTAATAGTGCTATTGGCAAAATTCAAGACTCACTTTCTTCCAC
AGCAAGTGCACTTGGAAAACTTCAAGATGTGGTCAACCAAAATGCACAAGCTTT
AAACACGCTTGTTAAACAACTTAGCTCCAATTTTGGTGCAATTTCAAGTGTTTTA
AATGATATCCTTTCACGTCTTGACAAAGTTGAGGCTGAAGTGCAAATTGATAGGT
TGATCACAGGCAGACTTCAAAGTTTGCAGACATATGTGACTCAACAATTAATTAG
AGCTGCAGAAATCAGAGCTTCTGCTAATCTTGCTGCTACTAAAATGTCAGAGTGT
GTACTTGGACAATCAAAAAGAGTTGATTTTTGTGGAAAGGGCTATCATCTTATGT
CCTTCCCTCAGTCAGCACCTCATGGTGTAGTCTTCTTGCATGTGACTTATGTCCCT
GCACAAGAAAAGAACTTCACAACTGCTCCTGCCATTTGTCATGATGGAAAAGCA
CACTTTCCTCGTGAAGGTGTCTTTGTTTCAAATGGCACACACTGGTTTGTAACAC
AAAGGAATTTTTATGAACCACAAATCATTACTACAGACAACACATTTGTGTCTGG
TAACTGTGATGTTGTAATAGGAATTGTCAACAACACAGTTTATGATCCTTTGCAA
CCTGAATTAGACTCATTCAAGGAGGAGTTAGATAAATATTTTAAGAATCATACAT
CACCAGATGTTGATTTAGGTGACATCTCTGGCATTAATGCTTCAGTTGTAAACAT
TCAAAAAGAAATTGACCGCCTCAATGAGGTTGCCAAGAATTTAAATGAATCTCTC
ATCGATCTCCAAGAACTTGGAAAGTATGAGCAGTATATAAAATGGCCATGGTAC
ATTTGGCTAGGTTTTATAGCTGGCTTGATTGCCATAGTAATGGTGACAATTATGCT
TTGCTGTATGACCAGTTGCTGTAGTTGTCTCAAGGGCTGTTGTTCTTGTGGATCCT
GCTGCAAATTTGATGAAGACGACTCTGAGCCAGTGCTCAAAGGAGTCAAATTAC
ATTACACATAA
-Spike protein encoded by SEQ ID NO: 1
SEQ ID NO: 2
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKG
VKLHYT

By “variant”, it is meant (1) a S gene from an isolate of SARS-CoV-2 other than the reference isolate; (2) a fragment of the S gene of SARS-CoV-2, or (3) a mutant of the S gene of SARS-CoV-2; including any combinations of the above. Additionally, “variant” also encompasses (1) a Spike protein from an isolate of SARS-CoV-2 other than the reference isolate; (2) a fragment of the Spike protein of SARS-CoV-2, or (3) a mutant of the Spike protein of SARS-CoV-2; including any combinations of the above.

In one embodiment, the S gene of SARS-CoV-2 coding for the Spike protein is from a SARS-CoV-2 isolate other than “Wuhan-Hu-1”.

By “fragment”, it is meant a nucleic acid sequence or an amino acid sequence comprising at least 70%, preferably at least 75%, 80%, 85%, 90%, 95%, 96%, 97%, 98%, 99% or more contiguous nucleotides or amino acid residues of the reference nucleic acid sequence or amino acid sequence.

In one embodiment, a fragment of the S gene of SARS-CoV-2 has a nucleic acid sequence coding for a Spike protein comprising at least 70%, preferably at least 75%, 80%, 85%, 90%, 95%, 96%, 97%, 98%, 99% or more contiguous amino acid residues of the amino acid sequence with SEQ ID NO: 2. In one embodiment, a fragment of the S gene of SARS-CoV-2 coding for the Spike protein comprises at least 70%, preferably at least 75%, 80%, 85%, 90%, 95%, 96%, 97%, 98%, 99% or more contiguous nucleotides of the nucleic acid sequence with SEQ ID NO: 1.

In one embodiment, a fragment of the S gene of SARS-CoV-2 has a nucleic acid sequence coding for a Spike protein which retains at least part or the entirety of its cytoplasmic domain, and the entirety of its transmembrane domain. In one embodiment, a fragment of the S gene of SARS-CoV-2 coding for the Spike protein retains at least part or the entirety of the cytoplasmic domain, and the entirety of the transmembrane domain of said Spike protein.

By “mutant”, it is meant a nucleic acid or amino acid sequence comprising at least 70%, preferably at least 75%, 80%, 85%, 90%, 95%, 96%, 97%, 98%, 99% or more sequence identity, preferably local sequence identity with the reference nucleic acid or amino acid sequence.

In one embodiment, a mutant of the S gene of SARS-CoV-2 has a nucleic acid sequence coding for a Spike protein comprising at least 70%, preferably at least 75%, 80%, 85%, 90%, 95%, 96%, 97%, 98%, 99% or more sequence identity, preferably local sequence identity with the amino acid sequence with SEQ ID NO: 2. In one embodiment, a mutant of the S gene of SARS-CoV-2 comprises at least 70%, preferably at least 75%, 80%, 85%, 90%, 95%, 96%, 97%, 98%, 99% or more sequence identity, preferably local sequence identity with the nucleic acid sequence with SEQ ID NO: 1.

Sequence identity refers to the number of identical or similar nucleotides or amino acid residues in a comparison between a test and a reference sequence. Sequence identity can be determined by sequence alignment of nucleic acid or amino acid sequences to identify regions of similarity or identity. For purposes herein, sequence identity is generally determined by alignment to identify identical nucleotides or amino acid residues. The alignment can be local or global. Matches, mismatches and gaps can be identified between compared sequences. Gaps are null nucleotides or amino acid residues inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as

$$\frac{number of identical positions}{length of the total aligned sequence} \times 100 .$$

A global alignment is an alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences, each of 100 nucleotides or amino acid residues in length, 50% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at deepc2.psi.iastate.edu/aat/align/align.html.

A local alignment is an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 50% sequence identity based on local alignment means that in an alignment of the full sequence of two compared sequences of any length, a region of similarity or identity of 100 nucleotides or amino acid residues in length has 50% of the residues that are the same in the region of similarity or identity.

For purposes herein, sequence identity can be determined by standard alignment algorithm programs used with default gap penalties established by each supplier. Default parameters for the GAP program can include:
(1) a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, D.C.: National Biomedical Research Foundation);
(2) a penalty of 3.0 for each gap and an additional 0.10 penalty for each symbol in each gap; and
(3) no penalty for end gaps.

Whether any mutant of the S gene of SARS-CoV-2 has a nucleic acid sequences that is at least 70%, preferably at least 75%, 80%, 85%, 90%, 95%, 96%, 97%, 98%, 99% or more “identical”, or other similar variations reciting a percent identity, can be determined using known computer algorithms based on local or global alignment (see, e.g., https://en.wikipedia.org/wiki/List_of_sequence_alignment_software [last accessed on Sep. 15, 2020], providing links to dozens of known and publicly available alignment databases and programs).

Generally, for purposes herein, sequence identity is determined using computer algorithms based on global alignment, such as the Needleman-Wunsch Global Sequence Alignment tool available from NCBI/BLAST (http://blast.ncbi.nlm.nih.gov/Blast.cgi); or LAlign (William Pearson implementing the Huang and Miller algorithm [Huang & Miller, 1991. Adv Appl Math. 12(3):337-57).

Typically, the full-length sequence of each of the compared nucleic acid or amino acid sequence is aligned across the full-length of each sequence in a global alignment. Local alignment also can be used when the sequences being compared are substantially the same length.

Therefore, the term identity represents a comparison or alignment between a test and a reference nucleic acid or amino acid sequence. In one exemplary embodiment, “at least 70% of sequence identity” refers to percent identities from 70 to 100% relative to the reference nucleic acid or amino acid sequence. Identity at a level of 70% or more is indicative of the fact that, assuming for exemplification purposes a test and reference nucleic acid or amino acid sequence length of 100 nucleotides or amino acid residues are compared, no more than 30 out of 100 nucleotides or amino acid residues in the test nucleic acid or amino acid sequence differ from those of the reference nucleic acid or amino acid sequence. Such differences can be represented as point mutations randomly distributed over the entire length of a nucleic acid or amino acid sequence or they can be clustered in one or more locations of varying length up to the maximum allowable, e.g., 30/100 nucleotide or amino acid residue difference (approximately 70% identity). Differences can also be due to deletions or truncations of nucleotides or amino acid residues. Differences are defined as nucleotide or amino acid residue substitutions, insertions or deletions. Depending on the length of the compared sequences, at the level of homologies or identities above about 85-90%, the result can be independent of the program and gap parameters set; such high levels of identity can be assessed readily, often without relying on software.

In one embodiment, a variant of the S gene of SARS-CoV-2 is a nucleic acid sequence with SEQ ID NO: 5. In one embodiment, a variant of the S gene of SARS-CoV-2 has a nucleic acid sequence coding for a Spike protein with SEQ ID NO: 6.

-Variant of the S gene
SEQ ID NO: 5
ATGGTGCTGCTGCTGATCCTGTCTGTGCTGCTCCTGAAAGAAGATGTGCGGG
GCTCTGCCCAGAGCACCGGTCAATGTGTGAACCTGACCACCAGAACACAGC
TGCCTCCAGCCTACACCAACAGCTTCACCAGAGGCGTGTACTACCCCGACA
AGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGC
ACCAAGAGATTCGACAACCCCGTGCTGCCCTTCAACGACGGGGTGTACTTT
GCCAGCACCGAGAAGTCCAACATCATCAGAGGCTGGATCTTCGGCACCACA
CTGGACAGCAAGACCCAGAGCCTGCTGATCGTGAACAACGCCACCAACGTG
GTCATCAAAGTGTGCGAGTTCCAGTTCTGCAACGACCCCTTCCTGGGCGTCT
ACTACCACAAGAACAACAAGAGCTGGATGGAAAGCGAGTTCCGGGTGTAC
AGCAGCGCCAACAACTGCACCTTCGAGTACGTGTCCCAGCCTTTCCTGATGG
ACCTGGAAGGCAAGCAGGGCAACTTCAAGAACCTGCGCGAGTTCGTGTTCA
AGAACATCGACGGCTACTTCAAGATCTACAGCAAGCACACCCCTATCAACC
TCGTGCGGGATCTGCCTCAGGGCTTCTCTGCTCTGGAACCCCTGGTGGATCT
GCCCATCGGCATCAACATCACCCGGTTTCAGACACTGCTGGCCCTGCACAG
AAGCTACCTGACACCTGGCGATAGCAGCTCTGGATGGACAGCTGGCGCCGC
TGCCTACTATGTGGGATACCTGCAGCCTCGGACCTTCCTGCTGAAGTACAAC
GAGAACGGCACCATCACCGACGCCGTGGATTGTGCTCTGGATCCTCTGAGC
GAGACAAAGTGCACCCTGAAGTCCTTCACCGTGGAAAAGGGCATCTACCAG
ACCAGCAACTTCCGGGTGCAGCCCACCGAATCCATCGTGCGGTTCCCCAAT
ATCACCAATCTGTGCCCCTTCGGCGAGGTGTTCAATGCCACCAGATTCGCCT
CTGTGTACGCCTGGAACCGGAAGCGGATCAGCAATTGCGTGGCCGACTACT
CCGTGCTGTACAACTCCGCCAGCTTCAGCACCTTCAAGTGCTACGGCGTGTC
CCCTACCAAGCTGAACGACCTGTGCTTCACAAACGTGTACGCCGACAGCTTC
GTGATCCGGGGAGATGAAGTGCGGCAGATTGCCCCTGGACAGACAGGCAA
GATCGCCGACTACAACTACAAGCTGCCCGACGACTTCACCGGCTGTGTGATT
GCCTGGAACAGCAACAACCTGGACTCCAAAGTCGGCGGCAACTACAATTAC
CTGTACCGGCTGTTCCGGAAGTCCAATCTGAAGCCCTTCGAGCGGGACATCT
CCACCGAGATCTATCAGGCCGGCAGCACCCCTTGTAACGGCGTGGAAGGCT
TCAACTGCTACTTCCCACTGCAGTCCTACGGCTTTCAGCCCACAAATGGCGT
GGGCTACCAGCCTTACAGAGTGGTGGTGCTGAGCTTCGAGCTGCTGCATGCT
CCTGCCACAGTGTGCGGCCCTAAGAAAAGCACCAATCTCGTGAAGAACAAA
TGCGTGAACTTCAACTTCAACGGCCTGACCGGCACCGGCGTGCTGACAGAG
AGCAACAAGAAGTTCCTGCCATTCCAGCAGTTCGGCCGGGATATCGCCGAT
ACCACAGATGCCGTCAGAGATCCCCAGACACTGGAAATCCTGGACATCACC
CCTTGCAGCTTCGGCGGAGTGTCTGTGATCACCCCTGGCACCAACACCAGCA
ATCAGGTGGCAGTGCTGTACCAGGACGTGAACTGTACCGAAGTGCCCGTGG
CCATTCACGCCGATCAGCTGACACCTACATGGCGGGTGTACTCCACCGGCA
GCAATGTGTTTCAGACCAGAGCCGGCTGTCTGATCGGAGCCGAGCACGTGA
ACAATAGCTACGAGTGCGACATCCCCATCGGCGCTGGCATCTGCGCCTCTTA
CCAGACACAGACAAACAGCCCCAGACGGGCCAGATCTGTGGCCAGCCAGA
GCATCATTGCCTACACAATGTCTCTGGGCGCCGAGAACAGCGTGGCCTACTC
CAACAACTCTATCGCTATCCCCACCAACTTCACCATCAGCGTGACCACAGAG
ATCCTGCCTGTGTCCATGACCAAGACCAGCGTGGACTGCACCATGTACATCT
GCGGCGATTCCACCGAGTGCTCCAACCTGCTGCTGCAGTACGGCAGCTTCTG
CACCCAGCTGAATAGAGCCCTGACAGGGATCGCCGTGGAACAGGATGCCAA
TACCGGCGAAGTGTTCGCCCAAGTGAAGCAGATCTACAAGACCCCTCCTAT
CAAGGACTTCGGCGGCTTCAATTTCAGCCAGATTCTGCCCGATCCTAGCAAG
CCCAGCAAGCGGAGCTTCATCGAGGACCTGCTGTTCAACAAAGTGACACTG
GCCGACGCCGGCTTCATCAAGCAGTATGGCGATTGTCTGGGCGACATTGCC
GCCAGGGATCTGATTTGCGCCCAGAAGTTTAACGGACTGACAGTGCTGCCT
CCTCTGCTGACCGATGAGATGATCGCCCAGTACACATCTGCCCTGCTGGCCG
GCACAATCACAAGCGGCTGGACATTTGGAGCTGGCGCTGCCCTGCAGATCC
CCTTTGCTATGCAGATGGCCTACAGATTCAACGGCATCGGAGTGACCCAGA
ATGTGCTGTACGAGAACCAGAAGCTGATCGCCAACCAGTTCAACAGCGCCA
TCGGCAAGATCCAGGACAGCCTGTCTAGCACAGCCAGCGCTCTGGGAAAGC
TGCAGGACGTGGTCAACCAGAATGCCCAGGCACTGAACACCCTGGTCAAGC
AGCTGAGCAGCAATTTCGGCGCCATCAGCTCTGTGCTGAACGATATCCTGA
GCAGACTGGACCCTCCTGAGGCCGAGGTGCAGATCGACAGACTGATCACCG
GAAGGCTGCAGTCCCTGCAGACCTACGTTACCCAGCAGCTGATCAGAGCCG
CCGAGATTAGAGCCTCTGCCAATCTGGCCGCCACCAAGATGTCTGAGTGTGT
GCTGGGCCAGAGCAAGAGAGTGGACTTTTGCGGCAAGGGCTACCACCTGAT
GAGCTTCCCTCAGTCTGCCCCTCACGGCGTGGTGTTTCTGCACGTGACATAC
GTGCCCGCTCAAGAGAAGAATTTCACCACCGCTCCAGCCATCTGCCACGAC
GGCAAAGCCCACTTTCCTAGAGAAGGCGTGTTCGTGTCCAACGGCACCCAT
TGGTTCGTGACCCAGCGGAACTTCTACGAGCCCCAGATCATCACCACCGAC
AACACCTTCGTGTCTGGCAACTGCGACGTCGTGATCGGCATTGTGAACAATA
CCGTGTACGACCCTCTGCAGCCCGAGCTGGACAGCTTCAAAGAGGAACTGG
ATAAGTACTTTAAGAACCACACAAGCCCCGACGTGGACCTGGGCGATATCA
GCGGAATCAATGCCAGCGTCGTGAACATCCAGAAAGAGATCGACCGGCTGA
ACGAGGTGGCCAAGAATCTGAACGAGAGCCTGATCGACCTGCAGGAACTGG
GGAAGTACGAGCAGTACATCAAGTGGCCCTGGTACATCTGGCTGGGCTTTA
TCGCCGGACTGATTGCCATCGTGATGGTCACAATCATGCTG
-Variant Spike protein encoded by SEQ ID NO: 5
SEQ ID NO: 6
MVLLLILSVLLLKEDVRGSAQSTG/QCVNLTTRTQLPPAYTNSFTRGVYYPDKV
PRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKS
WMESEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQ
TSNFRVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKKS
TNLVKNKCVNFNFNGLTGTGVLTESNKKFLPFQQFGRDIADTTDAVRDPQTLEI
LDITPCSFGGVSVITPGTNTSNQVAVLYQDVNCTEVPVAIHADQLTPTWRVYST
GSNVFQTRAGCLIGAEHVNNSYECDIPIGAGICASYQTQTNSPRRARSVASQSIIA
YTMSLGAENSVAYSNNSIAIPTNFTISVTTEILPVSMTKTSVDCTMYICGDSTECS
NLLLQYGSFCTQLNRALTGIAVEQDANTGEVFAQVKQIYKTPPIKDFGGFNFSQI
LPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQN
VLYENQKLIANQFNSAIGKIQDSLSSTASALGKLQDVVNQNAQALNTLVKQLSS
NFGAISSVLNDILSRLDPPEAEVQIDRLITGRLQSLQTYVTQQLIRAAEIRASANL
AATKMSECVLGQSKRVDFCGKGYHLMSFPQSAPHGVVFLHVTYVPAQEKNFT
TAPAICHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNIQKEIDR
LNEVAKNLNESLIDLQELGKYEQYIKWPWYIWLGFIAGLIAIVMVTIML

According to the present invention, the nucleic acid comprises a sequence coding for a pilot peptide which interacts with ESCRT proteins.

Pilot peptide which interacts with ESCRT proteins have been described in granted patents EP 2 268 816, and U.S. Pat. No. 9,546,371, which are incorporated by reference.

In one embodiment, the pilot peptide is capable of being addressed to the membrane vesicles, in particular to the exosome-forming vesicles, or to the cell compartment(s) involved in the formation of the membrane vesicles, and in particular the exosome-forming vesicles in eukaryotic cells. When integrated into a chimeric protein, such as a chimeric protein comprising an amino acid sequence of the Spike protein or any of the variants thereof defined hereinabove, fused or linked with said pilot peptide, the pilot peptide enables addressing said chimeric protein to the membrane vesicles and/or to their location(s) of formation and in particular to address said chimeric protein to the membrane of membrane vesicles, such that said protein can be secreted by a cell in association with the membrane vesicles (in particular exosomes), in particular an appropriate eukaryotic cell.

In one embodiment, the pilot peptide comprises at least one YxxL motif (SEQ ID NO: 17), in which “x” represents any amino acid residue. In particular, it may comprise one, two or three YxxL motifs with SEQ ID NO: 17.

In one embodiment, said YxxL motif or one of the YxxL motifs of the pilot peptide may, for example, be YINL (SEQ ID NO: 18) or YSHL (SEQ ID NO: 19).

In one embodiment, the pilot peptide comprises a DYxxL motif (SEQ ID NO: 20), in which “x” represents any residue.

In one embodiment, said DYxxL motif may, for example, be DYINL (SEQ ID NO: 21).

Alternatively or additionally, the pilot peptide comprises at least one motif equivalent to a YxxL motif (SEQ ID NO: 17), for example, a YxxF motif (SEQ ID NO: 22), in which “x” represents any residue.

Alternatively or additionally, the pilot peptide comprises at least one motif equivalent to a DYxxL motif (SEQ ID NO: 20), for example, a DYxxF motif (SEQ ID NO: 23), in which “x” represents any residue.

In one embodiment, the pilot peptide further comprises at least one PxxP motif (SEQ ID NO: 24), in which “x” represents any residue. In particular, it may comprise one, two, three or four PxxP motifs with SEQ ID NO: 24.

In one embodiment, said PxxP motif or one of the PxxP motifs of the pilot peptide is PSAP (SEQ ID NO: 25) or PTAP (SEQ ID NO: 26).

In one embodiment, the pilot peptide comprises at least one YxxL motif with SEQ ID NO: 17 or DYxxL motif with SEQ ID NO: 20, and at least one PxxP motif with SEQ ID NO: 24.

In one embodiment, the pilot peptide consists of an amino acid sequence having one, two or three YxxL motif(s) with SEQ ID NO: 17; and one, two, three or four PxxP motif(s) with SEQ ID NO: 24. In one embodiment, the pilot peptide consists of an amino acid sequence having three YxxL motifs with SEQ ID NO: 17; and four PxxP motifs with SEQ ID NO: 24.

In one embodiment, the YxxL motif with SEQ ID NO: 17 or one of the YxxL motifs with SEQ ID NO: 17 is located downstream, i.e., in a C-terminal position, with respect to the one or more PxxP motif(s) with SEQ ID NO: 24.

The proteins having a pilot peptide comprising at least one YxxL motif with SEQ ID NO: 17 include cellular proteins and viral proteins. In particular, these viral proteins are proteins of enveloped viruses, such as transmembrane glycoproteins of enveloped viruses, or herpesvirus proteins, e.g., the LMP2-A protein of the Epstein-Barr virus which comprises at least two YxxL motifs with SEQ ID NO: 17.

In one embodiment, the pilot peptide is that of a transmembrane glycoprotein of a retrovirus. In one embodiment, the pilot peptide may be that of a transmembrane glycoprotein of a retrovirus selected from the group comprising or consisting of bovine leukemia virus (BLV), human immunodeficiency virus (HIV) (such as, without limitation, HIV-1 or HIV-2), human T-cell leukemia virus (HTLV) (such as, without limitation, HTLV-1 or HTLV-2), and Mason-Pfizer monkey virus (MPMV).

In one embodiment, the pilot peptide comprises one of the following amino acid sequences:

SEQ ID NO: 27:
PxxPxxxxPxxPxSxYxxLxPxxPExYxxLxPxxPDYxxL;
SEQ ID NO: 28:
PxxPxₙPxxPxₙSxYxxLxₙPxxPExₙYxxLxₙPxxPDYxxL;
SEQ ID NO: 29:
PxxPxxxxPxxPxSxYxxLxPxxPExYxxLxPxxPDYxxLxxxx;
and
SEQ ID NO: 30:
PxxPxₙPxxPxₙSxYxxLxₙPxxPExₙYxxLxₙPxxPDYxxLxxxx;

in which “x” and “xₙ”, respectively, represent any residue and any one or several amino acid residue(s).

In one embodiment, the pilot peptide comprises one of the following amino acid sequences:

SEQ ID NO: 31:
PxxPxxxxxxxxxxxxYxxL;
SEQ ID NO: 32:
PxxPxxxxxxxxxxxDYxxL;
SEQ ID NO: 33:
PxxPxxYxxxxxxxxxYxxL;
SEQ ID NO: 34:
PxxPxxYxxxxxxxxDYxxL;
SEQ ID NO: 35:
PxxPExYxxLxPxxPDYxxL;
SEQ ID NO: 36:
PxxPxₙYxxL;
SEQ ID NO: 37:
PxxPxₙDYxxL;
SEQ ID NO: 38:
PxxPxₙYxₙYxxL;
SEQ ID NO: 39:
PxxPxₙYxₙDYxxL;
SEQ ID NO: 40:
PxxPExₙYxxLxₙPxxPDYxxL;
SEQ ID NO: 41:
PxxPxxxxPxxPxxxYxxLxPxxPExYxxLxPxxPDYxxL;
SEQ ID NO: 42:
PxxPxₙPxxPxₙYxxLxₙPxxPExₙYxxLxₙPxxPDYxxL;
SEQ ID NO: 43:
PxxPxxxxPxxPxxxYxxLxPxxPExYxxLxPxxPDYxxLxxxx;
and
SEQ ID NO: 44:
PxxPxₙPxxPxₙYxxLxₙPxxPExₙYxxLxₙPxxPDYxxLxxxx,

in which “x” and “xₙ”, respectively, represent any residue and any one or several amino acid residue(s).

In particular, “n” may be greater than or equal to 1 and less than 50. “n” may, in particular, have any value between 1 and 20.

In one embodiment, the pilot peptide comprises from 6 to 100 amino acid residues, in particular from 20 to 80, from 30 to 70, or from 40 to 60 amino acid residues, for example 40, 41, 42, 43, 44, 45, 46, 47, 48, 49 or 50 amino acid residues.

In one embodiment, the pilot peptide comprises or consists of an amino acid sequence with SEQ ID NO: 8 or a variant thereof. In one embodiment, the nucleic acid sequence coding for the pilot peptide with SEQ ID NO: 8 comprises or consists of SEQ ID NO: 7 or a variant thereof.

-CilPP
SEQ ID NO: 7
GCGCCCCACTTCCCTGAAATCTCCTTCCCCCCTAAACCCGATTCTGATT
ATCAGGCCTTGCTACCATCCGCGCCAGAGATCTACTCTCACCTCTCCCC
CACCAAACCCGATTACATCAACCTTCGACCGGCGCCCTAA
-CilPP encoded by SEQ ID NO: 7
SEQ ID NO: 8
APHFPEISFPPKPDSDYQALLPSAPEIYSHLSPTKPDYINLRPAP

In one embodiment, a variant of the amino acid sequence with SEQ ID NO: 8 comprises an amino acid sequence comprising at least 70%, preferably at least 75%, 80%, 85%, 90%, 95%, 96%, 97%, 98%, 99% or more contiguous amino acid residues of the amino acid sequence with SEQ ID NO: 8.

Additionally or alternatively, a variant of the amino acid sequence with SEQ ID NO: 8 comprises an amino acid sequence comprising at least 70%, preferably at least 75%, 80%, 85%, 90%, 95%, 96%, 97%, 98%, 99% or more sequence identity, preferably local sequence identity with the amino acid sequence with SEQ ID NO: 8.

In one embodiment, a variant of the amino acid sequence with SEQ ID NO: 8 retains at least one, two or three YxxL motif(s) with SEQ ID NO: 17 and at least one, two, three or four PxxP motif(s) with SEQ ID NO: 24. In one embodiment, a variant of the amino acid sequence with SEQ ID NO: 8 retains three YxxL motifs with SEQ ID NO: 17 and four PxxP motifs with SEQ ID NO: 24.

In one embodiment, the nucleic acid according to the present invention is inserted into a nucleic acid vector, preferably a nucleic acid expression vector.

In one embodiment, the nucleic acid according to the present invention is inserted into a nucleic acid vector, preferably a nucleic acid expression vector, operably linked to regulatory elements.

In one embodiment, the nucleic acid vector, preferably the nucleic acid expression vector, comprises, in this order, a promoter, the nucleic acid according to the present invention, a polyadenylation signal, and a termination sequence.

In one embodiment, the promoter is a cytomegalovirus-human T-lymphotropic virus type I (CMV-HTLV-I) chimeric promoter (Barouch et al., 2005. J Virol. 79(14):8828-8834).

In one embodiment, the polyadenylation signal and termination sequence is a bovine growth hormone (bGH) poly(A) signal (Pfarr et al., 1986. DNA. 5(2):115-122).

A further object of the present invention is an extracellular vesicle harboring at its external surface a Spike protein from a virus of the Orthocoronavirinae subfamily “S-EV”, or a variant thereof, as defined hereinabove.

The expression “harboring at its external surface” means that a Spike protein is anchored, through its transmembrane domain, in the lipid bilayer of the extracellular vesicle, and exposed, partially or completely, outside said membrane vesicle, included (completely or partially) in the membrane of said extracellular vesicle.

In one embodiment, the virus of the Orthocoronavirinae subfamily is a virus of the betacoronavirus genus, as defined hereinabove. In one embodiment, the virus of the Orthocoronavirinae subfamily is a virus of the betacoronavirus genus, and severe acute respiratory syndrome-related coronavirus species, as defined hereinabove. In one embodiment, the virus of the Orthocoronavirinae subfamily is a virus of the betacoronavirus genus, severe acute respiratory syndrome-related coronavirus species and strain SARS-CoV-2, as defined hereinabove. In one embodiment, the virus of the Orthocoronavirinae subfamily is SARS-CoV-2, as defined hereinabove.

In one embodiment, the Spike protein of SARS-CoV-2 has an amino acid sequence with SEQ ID NO: 2, or a variant thereof, as defined hereinabove.

Variants of the Spike protein of SARS-CoV-2 have been defined hereabove in the context of the nucleic acid “DNA^{S-EV}”, which applies here mutatis mutandis.

In one embodiment, the Spike protein is from a SARS-CoV-2 isolate other than “Wuhan-Hu-1”.

In one embodiment, a fragment of the Spike protein of SARS-CoV-2 comprises at least 70%, preferably at least 75%, 80%, 85%, 90%, 95%, 96%, 97%, 98%, 99% or more contiguous amino acid residues of the amino acid sequence with SEQ ID NO: 2.

In one embodiment, a fragment of the Spike protein of SARS-CoV-2 retains at least part or the entirety of its cytoplasmic domain, and the entirety of its transmembrane domain.

In one embodiment, a mutant of the Spike protein of SARS-CoV-2 comprises at least 70%, preferably at least 75%, 80%, 85%, 90%, 95%, 96%, 97%, 98%, 99% or more sequence identity, preferably local sequence identity with the amino acid sequence with SEQ ID NO: 2.

In one embodiment, a variant the Spike protein of SARS-CoV-2 comprises or consists of the amino acid sequence with SEQ ID NO: 6.

In one embodiment, the extracellular vesicle is an exosome.

In one embodiment, exosomes have a diameter ranging from about 30 nm to about 150 nm, preferably from about 30 nm to about 120 nm, more preferably from about 40 nm to about 80 nm.

A further object of the present invention is a population of extracellular vesicles harboring at their external surface a Spike protein from a virus of the Orthocoronavirinae subfamily “S-EV”, or a variant thereof, as defined hereinabove.

In one embodiment, the population of extracellular vesicles according to the present invention is monodisperse in aqueous solutions, preferably in water and/or in PBS.

By “monodisperse”, it is meant that the extracellular vesicles in the population of extracellular vesicles are substantially uniform in size. By “substantially uniform”, it is meant that the extracellular vesicles have a narrow distribution of sizes around an average size. In one embodiment, the extracellular vesicles in water and/or in PBS have sizes exhibiting a standard deviation of less than 100% with respect to their average size, such as less than 75%, 50%, 40%, 30%, 20%, 10%, or less than 5%.

A further object of the present invention is a method of obtaining an extracellular vesicle or a population of extracellular vesicles harboring at its/their external surface a Spike protein from a virus of the Orthocoronavirinae subfamily “S-EV”, or a variant thereof, as defined hereinabove.

General means and methods for obtaining extracellular vesicles or a population of extracellular vesicles are well known in the art. See, e.g., Whitford & Guterstam, 2019. Future Med Chem. 11(10):1225-1236; Taylor & Shah, 2015. Methods. 87:3-10; Desplantes et al., 2017. Sci Rep. 7(1):1032.

In one embodiment, the method of obtaining an extracellular vesicle or a population of extracellular vesicles comprises a step of producing the extracellular vesicle or the population of extracellular vesicles, as defined hereinabove.

In one embodiment, this step of producing the extracellular vesicle or the population of extracellular vesicles comprises transfecting cells with the nucleic acid “DNA^{S-EV}” comprising (i) a sequence of a S gene coding for a Spike protein from a virus of the Orthocoronavirinae subfamily, or a variant thereof; and (ii) a sequence coding for a pilot peptide which interacts with ESCRT proteins.

In one embodiment, the cells are HEK293 cells or cells from a derivative cell line. In one embodiment, the cells are immune cells, including, but not limited to, mastocytes, lymphocytes (such as, e.g., T-cells or B-cells), and dendritic cells.

In one embodiment, the method of obtaining an extracellular vesicle or a population of extracellular vesicles further comprises a step of culturing the transfected cells for a time sufficient to allow extracellular vesicle production, preferably in a medium devoid of EVs (i.e., a serum-free medium or a medium supplemented with EVs-depleted serum).

In one embodiment, the method of obtaining an extracellular vesicle or a population of extracellular vesicles further comprises a step of purifying said extracellular vesicle or population of extracellular vesicles.

In one embodiment, the step of purifying said extracellular vesicle or population of extracellular vesicles comprises clarification (such as, e.g., by centrifugation or by depth-filtration), filtration, ultra-filtration, diafiltration, size-exclusion purification and/or ion exchange chromatography of the transfected cell culture supernatant.

A further object of the present invention is a nucleic acid “DNA^{S-Trim}” comprising:
(i) a sequence of a S gene coding for a Spike protein from a virus of the Orthocoronavirinae subfamily, or a variant thereof; and
(ii) a sequence coding for a trimerization domain peptide.

In one embodiment, the virus of the Orthocoronavirinae subfamily is a virus of the betacoronavirus genus, as defined hereinabove. In one embodiment, the virus of the Orthocoronavirinae subfamily is a virus of the betacoronavirus genus, and severe acute respiratory syndrome-related coronavirus species, as defined hereinabove. In one embodiment, the virus of the Orthocoronavirinae subfamily is a virus of the betacoronavirus genus, severe acute respiratory syndrome-related coronavirus species and strain SARS-CoV-2, as defined hereinabove. In one embodiment, the virus of the Orthocoronavirinae subfamily is SARS-CoV-2, as defined hereinabove.

In one embodiment, the Spike protein of SARS-CoV-2 has an amino acid sequence with SEQ ID NO: 2, or a variant thereof, as defined hereinabove.

Variants of the Spike protein of SARS-CoV-2 have been defined hereabove in the context of the nucleic acid “DNA^{S-EV}”, which applies here mutatis mutandis.

In one embodiment, the Spike protein is from a SARS-CoV-2 isolate other than “Wuhan-Hu-1”.

In one embodiment, a fragment of the Spike protein of SARS-CoV-2 comprises at least 70%, preferably at least 75%, 80%, 85%, 90%, 95%, 96%, 97%, 98%, 99% or more contiguous amino acid residues of the amino acid sequence with SEQ ID NO: 2.

In one embodiment, a mutant of the Spike protein of SARS-CoV-2 comprises at least 70%, preferably at least 75%, 80%, 85%, 90%, 95%, 96%, 97%, 98%, 99% or more sequence identity, preferably local sequence identity with the amino acid sequence with SEQ ID NO: 2.

In one embodiment, a variant of the S gene of SARS-CoV-2 is a nucleic acid sequence with SEQ ID NO: 11. In one embodiment, a variant of the S gene of SARS-CoV-2 has a nucleic acid sequence coding for a Spike protein with SEQ ID NO: 12.

-Modified S gene
SEQ ID NO: 11
ATGGTGCTGCTGCTGATCCTGTCTGTGCTGCTCCTGAAAGAAGATGTGCGGG
GCTCTGCCCAGAGCACCGGTCAATGTGTGAACCTGACCACCAGAACACAGC
TGCCTCCAGCCTACACCAACAGCTTCACCAGAGGCGTGTACTACCCCGACA
AGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGC
ACCAAGAGATTCGACAACCCCGTGCTGCCCTTCAACGACGGGGTGTACTTT
GCCAGCACCGAGAAGTCCAACATCATCAGAGGCTGGATCTTCGGCACCACA
CTGGACAGCAAGACCCAGAGCCTGCTGATCGTGAACAACGCCACCAACGTG
GTCATCAAAGTGTGCGAGTTCCAGTTCTGCAACGACCCCTTCCTGGGCGTCT
ACTACCACAAGAACAACAAGAGCTGGATGGAAAGCGAGTTCCGGGTGTAC
AGCAGCGCCAACAACTGCACCTTCGAGTACGTGTCCCAGCCTTTCCTGATGG
ACCTGGAAGGCAAGCAGGGCAACTTCAAGAACCTGCGCGAGTTCGTGTTCA
AGAACATCGACGGCTACTTCAAGATCTACAGCAAGCACACCCCTATCAACC
TCGTGCGGGATCTGCCTCAGGGCTTCTCTGCTCTGGAACCCCTGGTGGATCT
GCCCATCGGCATCAACATCACCCGGTTTCAGACACTGCTGGCCCTGCACAG
AAGCTACCTGACACCTGGCGATAGCAGCTCTGGATGGACAGCTGGCGCCGC
TGCCTACTATGTGGGATACCTGCAGCCTCGGACCTTCCTGCTGAAGTACAAC
GAGAACGGCACCATCACCGACGCCGTGGATTGTGCTCTGGATCCTCTGAGC
GAGACAAAGTGCACCCTGAAGTCCTTCACCGTGGAAAAGGGCATCTACCAG
ACCAGCAACTTCCGGGTGCAGCCCACCGAATCCATCGTGCGGTTCCCCAAT
ATCACCAATCTGTGCCCCTTCGGCGAGGTGTTCAATGCCACCAGATTCGCCT
CTGTGTACGCCTGGAACCGGAAGCGGATCAGCAATTGCGTGGCCGACTACT
CCGTGCTGTACAACTCCGCCAGCTTCAGCACCTTCAAGTGCTACGGCGTGTC
CCCTACCAAGCTGAACGACCTGTGCTTCACAAACGTGTACGCCGACAGCTTC
GTGATCCGGGGAGATGAAGTGCGGCAGATTGCCCCTGGACAGACAGGCAA
GATCGCCGACTACAACTACAAGCTGCCCGACGACTTCACCGGCTGTGTGATT
GCCTGGAACAGCAACAACCTGGACTCCAAAGTCGGCGGCAACTACAATTAC
CTGTACCGGCTGTTCCGGAAGTCCAATCTGAAGCCCTTCGAGCGGGACATCT
CCACCGAGATCTATCAGGCCGGCAGCACCCCTTGTAACGGCGTGGAAGGCT
TCAACTGCTACTTCCCACTGCAGTCCTACGGCTTTCAGCCCACAAATGGCGT
GGGCTACCAGCCTTACAGAGTGGTGGTGCTGAGCTTCGAGCTGCTGCATGCT
CCTGCCACAGTGTGCGGCCCTAAGAAAAGCACCAATCTCGTGAAGAACAAA
TGCGTGAACTTCAACTTCAACGGCCTGACCGGCACCGGCGTGCTGACAGAG
AGCAACAAGAAGTTCCTGCCATTCCAGCAGTTCGGCCGGGATATCGCCGAT
ACCACAGATGCCGTCAGAGATCCCCAGACACTGGAAATCCTGGACATCACC
CCTTGCAGCTTCGGCGGAGTGTCTGTGATCACCCCTGGCACCAACACCAGCA
ATCAGGTGGCAGTGCTGTACCAGGACGTGAACTGTACCGAAGTGCCCGTGG
CCATTCACGCCGATCAGCTGACACCTACATGGCGGGTGTACTCCACCGGCA
GCAATGTGTTTCAGACCAGAGCCGGCTGTCTGATCGGAGCCGAGCACGTGA
ACAATAGCTACGAGTGCGACATCCCCATCGGCGCTGGCATCTGCGCCTCTTA
CCAGACACAGACAAACAGCCCCAGACGGGCCAGATCTGTGGCCAGCCAGA
GCATCATTGCCTACACAATGTCTCTGGGCGCCGAGAACAGCGTGGCCTACTC
CAACAACTCTATCGCTATCCCCACCAACTTCACCATCAGCGTGACCACAGAG
ATCCTGCCTGTGTCCATGACCAAGACCAGCGTGGACTGCACCATGTACATCT
GCGGCGATTCCACCGAGTGCTCCAACCTGCTGCTGCAGTACGGCAGCTTCTG
CACCCAGCTGAATAGAGCCCTGACAGGGATCGCCGTGGAACAGGATGCCAA
TACCGGCGAAGTGTTCGCCCAAGTGAAGCAGATCTACAAGACCCCTCCTAT
CAAGGACTTCGGCGGCTTCAATTTCAGCCAGATTCTGCCCGATCCTAGCAAG
CCCAGCAAGCGGAGCTTCATCGAGGACCTGCTGTTCAACAAAGTGACACTG
GCCGACGCCGGCTTCATCAAGCAGTATGGCGATTGTCTGGGCGACATTGCC
GCCAGGGATCTGATTTGCGCCCAGAAGTTTAACGGACTGACAGTGCTGCCT
CCTCTGCTGACCGATGAGATGATCGCCCAGTACACATCTGCCCTGCTGGCCG
GCACAATCACAAGCGGCTGGACATTTGGAGCTGGCGCTGCCCTGCAGATCC
CCTTTGCTATGCAGATGGCCTACAGATTCAACGGCATCGGAGTGACCCAGA
ATGTGCTGTACGAGAACCAGAAGCTGATCGCCAACCAGTTCAACAGCGCCA
TCGGCAAGATCCAGGACAGCCTGTCTAGCACAGCCAGCGCTCTGGGAAAGC
TGCAGGACGTGGTCAACCAGAATGCCCAGGCACTGAACACCCTGGTCAAGC
AGCTGAGCAGCAATTTCGGCGCCATCAGCTCTGTGCTGAACGATATCCTGA
GCAGACTGGACCCTCCTGAGGCCGAGGTGCAGATCGACAGACTGATCACCG
GAAGGCTGCAGTCCCTGCAGACCTACGTTACCCAGCAGCTGATCAGAGCCG
CCGAGATTAGAGCCTCTGCCAATCTGGCCGCCACCAAGATGTCTGAGTGTGT
GCTGGGCCAGAGCAAGAGAGTGGACTTTTGCGGCAAGGGCTACCACCTGAT
GAGCTTCCCTCAGTCTGCCCCTCACGGCGTGGTGTTTCTGCACGTGACATAC
GTGCCCGCTCAAGAGAAGAATTTCACCACCGCTCCAGCCATCTGCCACGAC
GGCAAAGCCCACTTTCCTAGAGAAGGCGTGTTCGTGTCCAACGGCACCCAT
TGGTTCGTGACCCAGCGGAACTTCTACGAGCCCCAGATCATCACCACCGAC
AACACCTTCGTGTCTGGCAACTGCGACGTCGTGATCGGCATTGTGAACAATA
CCGTGTACGACCCTCTGCAGCCCGAGCTGGACAGCTTCAAAGAGGAACTGG
ATAAGTACTTTAAGAACCACACAAGCCCCGACGTGGACCTGGGCGATATCA
GCGGAATCAATGCCAGCGTCGTGAACATCCAGAAAGAGATCGACCGGCTGA
ACGAGGTGGCCAAGAATCTGAACGAGAGCCTGATCGACCTGCAGGAACTGG
GGAAGTACGAGCAG
-Modified Spike protein encoded by SEQ ID NO: 11
SEQ ID NO: 12
MVLLLILSVLLLKEDVRGSAQSTGQCVNLTTRTQLPPAYTNSFTRGVYYPDKVFRSS
VLHSTQDLFLPFFSNVTWFHAIHVSGTNGTKRFDNPVLPFNDGVYFASTEKSNIIRGW
IFGTTLDSKTQSLLIVNNATNVVIKVCEFQFCNDPFLGVYYHKNNKSWMESEFRVYS
SANNCTFEYVSQPFLMDLEGKQGNFKNLREFVFKNIDGYFKIYSKHTPINLVRDLPQ
GFSALEPLVDLPIGINITRFQTLLALHRSYLTPGDSSSGWTAGAAAYYVGYLQPRTFL
LKYNENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQDVNCTEVP
VAIHADQLTPTWRVYSTGSNVFQTRAGCLIGAEHVNNSYECDIPIGAGICASYQTQT
NSPRRARSVASQSIIAYTMSLGAENSVAYSNNSIAIPTNFTISVTTEILPVSMTKTSVDC
TMYICGDSTECSNLLLQYGSFCTQLNRALTGIAVEQDANTGEVFAQVKQIYKTPPIKD
FGGFNFSQILPDPSKPSKRSFIEDLLFNKVTLADAGFIKQYGDCLGDIAARDLICAQKF
NGLTVLPPLLTDEMIAQYTSALLAGTITSGWTFGAGAALQIPFAMQMAYRFNGIGVT
QNVLYENQKLIANQFNSAIGKIQDSLSSTASALGKLQDVVNQNAQALNTLVKQLSSN
FGAISSVLNDILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTTAPAICHD
GKAHFPREGVFVSNGTHWFVTQRNFYEPQIITTDNTFVSGNCDVVIGIVNNTVYDPL
QPELDSFKEELDKYFKNHTSPDVDLGDISGINASVVNIQKEIDRLNEVAKNLNESLIDL
QELGKYEQ

In one embodiment, the trimerization domain peptide is selected from the group comprising or consisting of the T4-fibritin “Foldon” trimerization domain (Meier et al., 2004. J Mol Biol. 344(4):1051-1069; Güthe et al., 2004. J Mol Biol. 337(4):905-915), the GCN4-based isoleucine zipper (Drees et al., 1997. J Mol Biol. 273(1):61-74; Yang et al., 2000. J Virol. 74(12):5716-5725; Yang et al., 2002. J Virol. 76(9):4634-4642), the catalytic subunit of Escherichia coli aspartate transcarbamoylase (Chen et al., 2004. J Virol. 78(9):4508-4516), the HIV-1 gp41 trimerization domain (Shu et al., 1999. Biochemistry. 38(17):5378-5385), the SIV gp41 trimerization domain (Blacklow et al., 1995. Biochemistry. 34(46):14955-14962), the Ebola virus gp-2 trimerization domain, the HTLV-1 gp-21 trimerization domain, the yeast heat shock transcription factor trimerization domain, and the human collagen trimerization domain.

In one embodiment, the trimerization domain peptide is the T4-fibritin “Foldon” trimerization domain. In one embodiment, the sequence coding for a trimerization domain peptide is a sequence coding for the T4-fibritin “Foldon” trimerization domain.

In one embodiment, the T4-fibritin “Foldon” trimerization domain has an amino acid sequence with SEQ ID NO: 45.

SEQ ID NO: 45:
GYIPEAPRDGQAYVRKDGEWVLLSTFL

In one embodiment, the sequence coding for a trimerization domain peptide further codes for a cleavage site upstream of the trimerization domain peptide (i.e., in N-terminal position of the trimerization domain peptide).

In one embodiment, the protease is an endopeptidase. In one embodiment, the protease, preferably the endopeptidase, is a cysteine protease or a serine protease.

In one embodiment, the protease is selected from the group comprising or consisting of tobacco etch virus (TEV) protease, tobacco vein mottling virus (TVMV) protease, sugarcane mosaic virus (SMV) protease, hepatitis C virus (HCV) protease, pepsin, trypsin, chymotrypsin, thermolysin, subtilisin, papain, elastase, and plasminogen.

In one embodiment, the protease is TEV protease.

In one embodiment, the sequence coding for a trimerization domain peptide further codes for a purification tag downstream of the trimerization domain peptide (i.e., in C-terminal position of the trimerization domain peptide).

Examples of purification tags include, but are not limited to, poly-histidine tags (such as, e.g., 6×His or 10×His tags), avidin, FLAG-tag, glutathione S-transferase (GST) tag, maltose-binding protein (MBP) tag, chitin-binding protein (CBP) tag, V5-tag, Myc-tag, and HA-tags.

In one embodiment, the purification tag is a 6×His tag.

In one embodiment, the sequence coding for the trimerization domain peptide has a nucleic acid sequence coding for a cleavage site, a linker, a trimerization domain peptide and a purification tag, with an amino acid sequence with SEQ ID NO: 14. In one embodiment, the sequence coding for the trimerization domain peptide has a nucleic acid sequence with SEQ ID NO: 13.

-Trimerization foldon
SEQ ID NO: 13
AGCGGCAGAGAGAACCTGTACTTCCAAGGCGGAGGCGGCGGAAGCGGCT
ATATTCCTGAAGCTCCTAGAGATGGCCAGGCCTACGTGCGGAAAGATGG
CGAATGGGTCCTGCTGAGCACCTTTCTGGGACACCACCACCATCACCAT
TAGTAA
-Trimerization foldon encoded by SEQ ID NO: 13
SEQ ID NO: 14
SGRENLYFQGGGGGSGYIPEAPRDGQAYVRKDGEWVLLSTFLGHHHHHH

A further object of the present invention is a trimeric Spike protein from a virus of the Orthocoronavirinae subfamily “S-Trim”, or a variant thereof, as defined hereinabove.

In one embodiment, the virus of the Orthocoronavirinae subfamily is a virus of the betacoronavirus genus, as defined hereinabove. In one embodiment, the virus of the Orthocoronavirinae subfamily is a virus of the betacoronavirus genus, and severe acute respiratory syndrome-related coronavirus species, as defined hereinabove. In one embodiment, the virus of the Orthocoronavirinae subfamily is a virus of the betacoronavirus genus, severe acute respiratory syndrome-related coronavirus species and strain SARS-CoV-2, as defined hereinabove. In one embodiment, the virus of the Orthocoronavirinae subfamily is SARS-CoV-2, as defined hereinabove.

In one embodiment, each monomer of the trimeric Spike protein of SARS-CoV-2 has an amino acid sequence with SEQ ID NO: 2, or a variant thereof, as defined hereinabove.

Variants of the Spike protein of SARS-CoV-2 have been defined hereabove in the context of the nucleic acid “DNA^{S-EV}”, which applies here mutatis mutandis.

In one embodiment, the Spike protein is from a SARS-CoV-2 isolate other than “Wuhan-Hu-1”.

In one embodiment, a fragment of the Spike protein of SARS-CoV-2 comprises at least 70%, preferably at least 75%, 80%, 85%, 90%, 95%, 96%, 97%, 98%, 99% or more contiguous amino acid residues of the amino acid sequence with SEQ ID NO: 2.

In one embodiment, a fragment of the Spike protein of SARS-CoV-2 retains at least part or the entirety of its cytoplasmic domain, and the entirety of its transmembrane domain.

In one embodiment, a mutant of the Spike protein of SARS-CoV-2 comprises at least 70%, preferably at least 75%, 80%, 85%, 90%, 95%, 96%, 97%, 98%, 99% or more sequence identity, preferably local sequence identity with the amino acid sequence with SEQ ID NO: 2.

In one embodiment, a variant of a monomer of the trimeric Spike protein of SARS-CoV-2 comprises or consists of the amino acid sequence with SEQ ID NO: 12.

In one embodiment, a variant of a monomer of the trimeric Spike protein of SARS-CoV-2 comprises or consists of the amino acid sequences with SEQ ID NO: 12 and SEQ ID NO: 14. In one embodiment, a variant of a monomer of the trimeric Spike protein of SARS-CoV-2 comprises or consists, for N- to C-terminal, of the amino acid sequence with SEQ ID NO: 12 and the amino acid sequence with SEQ ID NO: 14.

In one embodiment, the trimeric Spike protein is isolated or otherwise purified.

A further object of the present invention is a method of obtaining a trimeric Spike protein from a virus of the Orthocoronavirinae subfamily “S-Trim”, or a variant thereof, as defined hereinabove.

In one embodiment, the method of obtaining a trimeric Spike protein comprises a step of producing the trimeric Spike protein, as defined hereinabove.

In one embodiment, this step of producing the trimeric Spike protein comprises transfecting cells with the nucleic acid “DNA^{S-Trim}” comprising (i) a sequence of a S gene coding for a Spike protein from a virus of the Orthocoronavirinae subfamily, or a variant thereof, and (ii) a sequence coding for a trimerization domain peptide.

In one embodiment, the cells are HEK293 cells or cells from a derivative cell line.

In one embodiment, the method of obtaining a trimeric Spike protein further comprises a step of culturing the transfected cells for a time sufficient to allow Spike protein production.

In one embodiment, the method of obtaining a trimeric Spike protein further comprises a step of purifying said trimeric Spike protein.

In one embodiment, the step of purifying said trimeric Spike protein comprises lysing host cells, centrifuging and/or affinity purifying.

In particular, where the sequence coding for a trimerization domain peptide further codes for a purification tag in the nucleic acid “DNA^{S-Trim}”, this purification tag can serve for affinity purifying.

A further object of the invention is a composition comprising a nucleic acid “DNA^{S-EV} ” comprising:
(i) a sequence of a S gene coding for a Spike protein from a virus of the Orthocoronavirinae subfamily, or a variant thereof; and
(ii) a sequence coding for a pilot peptide which interacts with ESCRT proteins as defined hereinabove.

A further object of the invention is a composition comprising an extracellular vesicle harboring at its external surface a Spike protein from a virus of the Orthocoronavirinae subfamily “S-EV”, or a variant thereof, as defined hereinabove.

A further object of the invention is a composition comprising a population of extracellular vesicles harboring at their external surface a Spike protein from a virus of the Orthocoronavirinae subfamily “S-EV”, or a variant thereof, as defined hereinabove.

A further object of the invention is a composition comprising a nucleic acid “DNA^{S-Trim} ” comprising:
(i) a sequence of a S gene coding for a Spike protein from a virus of the Orthocoronavirinae subfamily, or a variant thereof; and
(ii) a sequence coding for a trimerization domain peptide, as defined hereinabove.

A further object of the invention is a composition comprising a trimeric Spike protein from a virus of the Orthocoronavirinae subfamily “S-Trim”, or a variant thereof, as defined hereinabove.

In one embodiment, the compositions according to the present invention are pharmaceutical composition and further comprise at least one pharmaceutically acceptable excipient.

The term “pharmaceutically acceptable excipient” includes any and all solvents, diluents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of vegetable oil saturated fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical compositions comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

In one embodiment, the compositions according to the present invention do not comprise any adjuvant.

Examples of adjuvants include, but are not limited to, cholera toxin, Escherichia coli heat-labile enterotoxin (LT), liposome, immune-stimulating complex (ISCOM), immunostimulatory sequences oligodeoxynucleotide (ISS-ODN), aluminum salts (such as, e.g., aluminum hydroxide or aluminum phosphate), Freund's complete adjuvant, Freund's incomplete adjuvant, Ribi solution, Corynebacterium parvum, Bacillus Calmette-Guérin (BCG), glucan, dextran sulfate, iron oxide, sodium alginate, and muramyl peptides.

A further object is a kit-of-parts comprising:
(i) a nucleic acid “DNA^{S-EV}” comprising a sequence of a S gene coding for a Spike protein from a virus of the Orthocoronavirinae subfamily, or a variant thereof; and a sequence coding for a pilot peptide which interacts with ESCRT proteins, as defined hereinabove; or a composition or pharmaceutical composition comprising the same; and
(ii) either of:
   a. an extracellular vesicle harboring at its external surface a Spike protein from a virus of the Orthocoronavirinae subfamily “S-EV”, or a variant thereof, as defined hereinabove; or a composition or pharmaceutical composition comprising the same;
   b. a population of extracellular vesicles harboring at their external surface a Spike protein from a virus of the Orthocoronavirinae subfamily “S-EV”, or a variant thereof, as defined hereinabove; or a composition or pharmaceutical composition comprising the same; or
   c. a trimeric Spike protein from a virus of the Orthocoronavirinae subfamily “S-Trim”, or a variant thereof, as defined hereinabove; or a composition or pharmaceutical composition comprising the same.

A further object of the present invention is a method of immunizing a subject against a virus of the Orthocoronavirinae subfamily.

In one embodiment, the virus of the Orthocoronavirinae subfamily is a virus of the betacoronavirus genus, as defined hereinabove. In one embodiment, the virus of the Orthocoronavirinae subfamily is a virus of the betacoronavirus genus, and severe acute respiratory syndrome-related coronavirus species, as defined hereinabove. In one embodiment, the virus of the Orthocoronavirinae subfamily is a virus of the betacoronavirus genus, severe acute respiratory syndrome-related coronavirus species and strain SARS-CoV-2, as defined hereinabove. In one embodiment, the virus of the Orthocoronavirinae subfamily is SARS-CoV-2, as defined hereinabove.

In one embodiment, the method of immunizing a subject against a virus of the Orthocoronavirinae subfamily comprises a first step of administering to said subject a nucleic acid “DNA^{S-EV}” comprising a sequence of a S gene coding for a Spike protein from a virus of the Orthocoronavirinae subfamily, or a variant thereof; and a sequence coding for a pilot peptide which interacts with ESCRT proteins, as defined hereinabove; or a composition or pharmaceutical composition comprising the same.

Means and methods for administering a nucleic acid to a subject (i.e., for DNA vaccine delivery) are well known in the art. Any convenient and appropriate method of delivery of the nucleic acid may be utilized. These include, e.g., delivery with cationic lipids (Goddard et al., 1997. Gene Ther. 4(11):1231-1236; Gorman et al., 1997. Gene Ther. 4(9):983-992; Chadwick et al., 1997. Gene Ther. 4(9):937-942; Gokhale et al., 1997. Gene Ther. 4(12):1289-1299; Gao & Huang, 1995. Gene Ther. 2(10):710-722), delivery by uptake of naked nucleic acid, and the like. Method of delivery of the nucleic acid can further include or be enhanced by electroporation, particle bombardment, sonoporation, magnetofection, hydrodynamic delivery and the like. Method of delivery of the nucleic acid can further include or be enhanced by the use of chemicals including, but not limited to, nucleic acid specifically modified to enhance delivery, lipoplexes, polymersomes, polyplexes, dendrimers, nanoparticles (e.g., inorganic nanoparticles), cell-penetrating peptides, cell-penetrating proteins (e.g., supercharged proteins), and the like.

In one embodiment, the method of immunizing a subject against a virus of the Orthocoronavirinae subfamily comprises a second step of administering to said subject either of:
a. an extracellular vesicle harboring at its external surface a Spike protein from a virus of the Orthocoronavirinae subfamily “S-EV”, or a variant thereof, as defined hereinabove; or a composition or pharmaceutical composition comprising the same;
b. a population of extracellular vesicles harboring at their external surface a Spike protein from a virus of the Orthocoronavirinae subfamily “S-EV”, or a variant thereof, as defined hereinabove; or a composition or pharmaceutical composition comprising the same; or
c. a trimeric Spike protein from a virus of the Orthocoronavirinae subfamily “S-Trim”, or a variant thereof, as defined hereinabove; or a composition or pharmaceutical composition comprising the same.

In one embodiment, the method of immunizing a subject against a virus of the Orthocoronavirinae subfamily comprises:
1) a first step, termed “priming step”, of administering to said subject a nucleic acid “DNA^{S-EV}” comprising a sequence of a S gene coding for a Spike protein from a virus of the Orthocoronavirinae subfamily, or a variant thereof; and a sequence coding for a pilot peptide which interacts with ESCRT proteins, as defined hereinabove; or a composition or pharmaceutical composition comprising the same; and
2) a second step, termed “boosting step”, of administering to said subject either of:
   a. an extracellular vesicle harboring at its external surface a Spike protein from a virus of the Orthocoronavirinae subfamily “S-EV”, or a variant thereof, as defined hereinabove; or a composition or pharmaceutical composition comprising the same;
   b. a population of extracellular vesicles harboring at their external surface a Spike protein from a virus of the Orthocoronavirinae subfamily “S-EV”, or a variant thereof, as defined hereinabove; or a composition or pharmaceutical composition comprising the same; or
   c. a trimeric Spike protein from a virus of the Orthocoronavirinae subfamily “S-Trim”, or a variant thereof, as defined hereinabove; or a composition or pharmaceutical composition comprising the same.

In one embodiment, the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same are formulated for administration to the subject.

In one embodiment, the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same is/are to be administered systemically or locally.

In one embodiment, the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same is/are to be administered by injection, orally, topically, nasally, buccally, rectally, vaginaly, intratracheally, by endoscopy, transmucosally, or by percutaneous administration.

In one embodiment, the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same is/are to be injected, preferably systemically injected.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv) injection, subcutaneous injection, intramuscular (im) injection, intradermal (id) injection, intraperitoneal (ip), intranasal (in) injection and perfusion.

It will be understood that other suitable routes of administration are also contemplated in the present invention, and the administration mode will ultimately be decided by the attending physician within the scope of sound medical judgment.

In one embodiment, the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same is/are to be administered to the subject in need thereof in a therapeutically effective amount.

The term “therapeutically effective amount” or “therapeutically effective dose”, as used herein, refers to an amount effective, at dosages and for periods of time necessary, to achieve a desired preventive result. In particular, a therapeutically effective amount refers to an amount effective, at dosages and for periods of time necessary, to achieve (1) a humoral immune response as can be determined, e.g., by increased antibody titers against the “head” S1 and the “stem” S2 subunits of the Spike protein from the virus of the Orthocoronavirinae subfamily, or a variant thereof; and/or (2) a T-cell mediated immunity for the “head” S1 and the “stem” S2 subunits of the Spike protein from the virus of the Orthocoronavirinae subfamily, or a variant thereof; and/or (3) “head” S1 and “stem” S2 subunits-specific interferon (IFN)-γ production.

It will be however understood that the dosage of the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same will be decided by the attending physician within the scope of sound medical judgment. The specific therapeutically effective dose for any particular patient will depend upon a variety of factors including the disease being prevented and the severity of the disease; the activity of the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same employed; the age, body weight, general health, sex and diet of the subject; the time of administration, route of administration, and rate of excretion of the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same employed; the duration and regimen of the treatment; drugs used in combination or coincidental with the nucleic acid “DNA^{S-EV}” or the composition or pharmaceutical composition comprising the same, the extracellular vesicle “S-EV” or the population of extracellular vesicles “S-EV” or the composition or pharmaceutical composition comprising the same, and/or the trimeric Spike protein “S-Trim” or the composition or pharmaceutical composition comprising the same employed; and like factors well known in the medical arts.

In one embodiment, the “priming step” is to be carried out once, twice, three times, four times or more. In one embodiment, the “boosting step” is to be carried out once, twice, three times, four times or more.

In one embodiment, the “priming step” is to be carried out twice. In one embodiment, the “boosting step” is to be carried out once.

In one embodiment, the “priming step” is to be carried out twice and the “boosting step” is to be carried out once.

In one embodiment, the period of time between each iteration of the “priming step” and/or between each iteration of the “boosting step” and/or between the “priming step” and the “boosting step” range from about 1 day to about 6 months, preferably from about 1 week to about 3 months, more preferably from about 2 weeks to about 1 month.

In one embodiment, the period of time between each iteration of the “priming step” and/or between each iteration of the “boosting step” and/or between the “priming step” and the “boosting step” is about 1 day, 2 days, 3 days, 4 days, 5 days, 6 days, 1 week, 2 weeks, 3 weeks, 1 month, 2 months, 3 months, 4 months, 5 months, or 6 months.

In one embodiment, the period of time between each iteration of the “priming step” and/or between each iteration of the “boosting step” and/or between the “priming step” and the “boosting step” is about 3 weeks.

Therapeutically effective human doses can be calculated from animal doses, by converting these doses to Human Equivalent Doses (HED) based on body surface area (BSA). According to the FDA “Guidance for Industry” of July 2005, the BSA of a human with a body weight of 60 kg is about 1.62 m², while the BSA of a mouse (such as, e.g., Balb/c mice used in the experimental design described below) with a body weight of 0.020 kg is about 0.007 m². To convert mouse dose in μg/kg to HED in μg/kg, the mouse dose can be divided by 12.3 (or alternatively multiplied by 0.081).

Additionally or alternatively, one skilled in the art is well aware that therapeutically effective human doses of nucleic acids for DNA vaccination typically range between about 0.1 mg and about 6 mg of nucleic acid per administration, with some studies describing doses up to about 10 mg of nucleic acid per administration.

In one embodiment, a therapeutically effective human dose of the nucleic acid “DNA^{S-EV}” or the composition or pharmaceutical composition comprising the same to be administered during the—or each one of the—“priming step” ranges from about 0.1 μg/kg to about 50 μg/kg of nucleic acid.

In one embodiment, a therapeutically effective human dose of the nucleic acid “DNA^{S-EV}” or the composition or pharmaceutical composition comprising the same to be administered during the—or each one of the—“priming step” is about 0.1 μg/kg±0.05 μg/kg, 0.2 μg/kg±0.05 μg/kg, 0.3 μg/kg±0.05 μg/kg, 0.4 μg/kg±0.05 μg/kg, 0.5 μg/kg±0.05 μg/kg, 0.6 μg/kg±0.05 μg/kg, 0.7 μg/kg±0.05 μg/kg, 0.8 μg/kg±0.05 μg/kg, 0.9 μg/kg±0.05 μg/kg, 1 μg/kg±0.5 μg/kg, 2 μg/kg±0.5 μg/kg, 3 μg/kg±0.5 μg/kg, 4 μg/kg±0.5 μg/kg, 5 μg/kg±0.5 μg/kg, 6 μg/kg±0.5 μg/kg, 7 μg/kg±0.5 μg/kg, 8 μg/kg±0.5 μg/kg, 9 μg/kg±0.5 μg/kg, 10 μg/kg±0.5 μg/kg, 11 μg/kg±0.5 μg/kg, 12 μg/kg±0.5 μg/kg, 13 μg/kg±0.5 μg/kg, 14 μg/kg±0.5 μg/kg, 15 μg/kg±0.5 μg/kg, 16 μg/kg±0.5 μg/kg, 17 μg/kg±0.5 μg/kg, 18 μg/kg±0.5 μg/kg, 19 μg/kg±0.5 μg/kg, 20 μg/kg±0.5 μg/kg, 21 μg/kg±0.5 μg/kg, 22 μg/kg±0.5 μg/kg, 23 μg/kg±0.5 μg/kg, 24 μg/kg±0.5 μg/kg, 25 μg/kg±0.5 μg/kg, 26 μg/kg±0.5 μg/kg, 27 μg/kg±0.5 μg/kg, 28 μg/kg±0.5 μg/kg, 29 μg/kg±0.5 μg/kg, 30 μg/kg±0.5 μg/kg, 31 μg/kg±0.5 μg/kg, 32 μg/kg±0.5 μg/kg, 33 μg/kg±0.5 μg/kg, 34 μg/kg±0.5 μg/kg, 35 μg/kg±0.5 μg/kg, 36 μg/kg±0.5 μg/kg, 37 μg/kg±0.5 μg/kg, 38 μg/kg±0.5 μg/kg, 39 μg/kg±0.5 μg/kg, 40 μg/kg±0.5 μg/kg, 41 μg/kg±0.5 μg/kg, 42 μg/kg±0.5 μg/kg, 43 μg/kg±0.5 μg/kg, 44 μg/kg±0.5 μg/kg, 45 μg/kg±0.5 μg/kg, 46 μg/kg±0.5 μg/kg, 47 μg/kg±0.5 μg/kg, 48 μg/kg±0.5 μg/kg, 49 μg/kg±0.5 μg/kg, or 50 μg/kg±0.5 μg/kg of nucleic acid.

In one embodiment, a therapeutically effective human dose of the extracellular vesicle “S-EV” or the population of extracellular vesicles “S-EV” or the composition or pharmaceutical composition comprising the same to be administered during the—or each one of the—“boosting step” ranges from about 0.1 μg/kg to about 50 μg/kg of extracellular vesicle.

In one embodiment, a therapeutically effective human dose of the extracellular vesicle “S-EV” or the population of extracellular vesicles “S-EV” or the composition or pharmaceutical composition comprising the same to be administered during the—or each one of the—“boosting step” is about 0.1 μg/kg±0.05 μg/kg, 0.2 μg/kg±0.05 μg/kg, 0.3 μg/kg±0.05 μg/kg, 0.4 μg/kg±0.05 μg/kg, 0.5 μg/kg±0.05 μg/kg, 0.6 μg/kg±0.05 μg/kg, 0.7 μg/kg±0.05 μg/kg, 0.8 μg/kg±0.05 μg/kg, 0.9 μg/kg±0.05 μg/kg, 1 μg/kg±0.5 μg/kg, 2 μg/kg±0.5 μg/kg, 3 μg/kg±0.5 μg/kg, 4 μg/kg±0.5 μg/kg, 5 μg/kg±0.5 μg/kg, 6 μg/kg±0.5 μg/kg, 7 μg/kg±0.5 μg/kg, 8 μg/kg±0.5 μg/kg, 9 μg/kg±0.5 μg/kg, 10 μg/kg±0.5 μg/kg, 11 μg/kg±0.5 μg/kg, 12 μg/kg±0.5 μg/kg, 13 μg/kg±0.5 μg/kg, 14 μg/kg±0.5 μg/kg, 15 μg/kg±0.5 μg/kg, 16 μg/kg±0.5 μg/kg, 17 μg/kg±0.5 μg/kg, 18 μg/kg±0.5 μg/kg, 19 μg/kg±0.5 μg/kg, 20 μg/kg±0.5 μg/kg, 21 μg/kg±0.5 μg/kg, 22 μg/kg±0.5 μg/kg, 23 μg/kg±0.5 μg/kg, 24 μg/kg±0.5 μg/kg, 25 μg/kg±0.5 μg/kg, 26 μg/kg±0.5 μg/kg, 27 μg/kg±0.5 μg/kg, 28 μg/kg±0.5 μg/kg, 29 μg/kg±0.5 μg/kg, 30 μg/kg±0.5 μg/kg, 31 μg/kg±0.5 μg/kg, 32 μg/kg±0.5 μg/kg, 33 μg/kg±0.5 μg/kg, 34 μg/kg±0.5 μg/kg, 35 μg/kg±0.5 μg/kg, 36 μg/kg±0.5 μg/kg, 37 μg/kg±0.5 μg/kg, 38 μg/kg±0.5 μg/kg, 39 μg/kg±0.5 μg/kg, 40 μg/kg±0.5 μg/kg, 41 μg/kg±0.5 μg/kg, 42 μg/kg±0.5 μg/kg, 43 μg/kg±0.5 μg/kg, 44 μg/kg±0.5 μg/kg, 45 μg/kg±0.5 μg/kg, 46 μg/kg±0.5 μg/kg, 47 μg/kg±0.5 μg/kg, 48 μg/kg±0.5 μg/kg, 49 μg/kg±0.5 μg/kg, 50 μg/kg±0.5 μg/kg, of extracellular vesicle.

A further object of the present invention is an isolated peptide targeting the membrane proximal external region (MPER) subdomain of the Spike protein from a virus of the Orthocoronavirinae subfamily, or a variant thereof, as defined hereinabove.

In one embodiment, the virus of the Orthocoronavirinae subfamily is a virus of the betacoronavirus genus, as defined hereinabove. In one embodiment, the virus of the Orthocoronavirinae subfamily is a virus of the betacoronavirus genus, and severe acute respiratory syndrome-related coronavirus species, as defined hereinabove. In one embodiment, the virus of the Orthocoronavirinae subfamily is a virus of the betacoronavirus genus, severe acute respiratory syndrome-related coronavirus species and strain SARS-CoV-2, as defined hereinabove. In one embodiment, the virus of the Orthocoronavirinae subfamily is SARS-CoV-2, as defined hereinabove.

In one embodiment, the Spike protein of SARS-CoV-2 has an amino acid sequence with SEQ ID NO: 2, or a variant thereof, as defined hereinabove.

Variants of the Spike protein of SARS-CoV-2 have been defined hereabove in the context of the nucleic acid “DNA^{S-EV}”, which applies here mutatis mutandis.

In one embodiment, the Spike protein is from a SARS-CoV-2 isolate other than “Wuhan-Hu-1”.

In one embodiment, a fragment of the Spike protein of SARS-CoV-2 comprises at least 70%, preferably at least 75%, 80%, 85%, 90%, 95%, 96%, 97%, 98%, 99% or more contiguous amino acid residues of the amino acid sequence with SEQ ID NO: 2.

In one embodiment, a mutant of the Spike protein of SARS-CoV-2 comprises at least 70%, preferably at least 75%, 80%, 85%, 90%, 95%, 96%, 97%, 98%, 99% or more sequence identity, preferably local sequence identity with the amino acid sequence with SEQ ID NO: 2.

In one embodiment, the isolated peptide targeting the MPER subdomain of the Spike protein comprises or consists of an amino acid sequence comprising or consisting of the C-terminal part of the extracellular domain of said Spike protein. The extracellular domain of the Spike protein of SARS-CoV-2 with SEQ ID NO: 2 comprises amino acid residues 13 to 1217 of SEQ ID NO: 2.

In one embodiment, the isolated peptide targeting the MPER subdomain of the Spike protein comprises or consists of an amino acid sequence comprising or consisting of 5 to 50 amino acid residues of the C-terminal part of the extracellular domain of said Spike protein, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acid residues.

In one embodiment, the isolated peptide targeting the MPER subdomain of the Spike protein comprises or consists of an amino acid sequence selected from SEQ ID NO: 46 to SEQ ID NO: 56.

SEQ ID NO: 46:
PWYIW
SEQ ID NO: 47:
WPWYIW
SEQ ID NO: 48:
KWPWYIW
SEQ ID NO: 49:
IKWPWYIW
SEQ ID NO: 50:
YIKWPWYIW
SEQ ID NO: 51:
QYIKWPWYIW
SEQ ID NO: 52:
EQYIKWPWYIW
SEQ ID NO: 53:
YEQYIKWPWYIW
SEQ ID NO: 54:
KYEQYIKWPWYIW
SEQ ID NO: 55:
GKYEQYIKWPWYIW
SEQ ID NO: 56:
LGKYEQYIKWPWYIW

In one embodiment, the isolated peptide targeting the MPER subdomain of the Spike protein does not comprises or consists of the amino acid sequence of the “HR2 region”. By “HR2 region”, it is meant the heptad repeat 2 region of the Spike protein. The HR2 region of the Spike protein of SARS-CoV-2 with SEQ ID NO: 2 comprises amino acid residues 1163 to 1202 of SEQ ID NO: 2.

When reciting “the isolated peptide targeting the MPER subdomain of the Spike protein”, also encompassed are isolated peptide variants, such as peptides of a variant Spike protein (as previously defined), peptidomimetics, conjugates, and the like, including any combinations thereof.

In one embodiment, a conjugate of the isolated peptide targeting the MPER subdomain of the Spike protein comprises said isolated peptide, fused or linked to a lipid moiety. In particular, such lipid moiety allows to associate the isolated peptide with the external surface of a cell or a virus, by, e.g., anchoring said isolated peptide to the lipid bilayer. In this respect, the lipid moiety is termed “hydrophobic anchor”.

Typical hydrophobic anchors include, but are not limited to, acyl (such as, e.g., myristoyl or palmitoyl) and prenyl (such as, e.g., linear poly-isoprene) chains.

In one embodiment, peptidomimetics of the isolated peptide targeting the MPER subdomain of the Spike protein comprise, but are not limited to, retro analogues, inverso analogues and retroinverso analogues of said isolated peptide.

In one embodiment, a conjugate of the isolated peptide targeting the MPER subdomain of the Spike protein comprises or consists of said isolated peptide fused to a payload, such as, a therapeutic agent, a diagnostic agent, or a carrier. In particular, carriers can improve the pharmacokinetics and/or biodistribution of the isolated peptide. Such conjugates are sometimes referred to as “biobetters”. For a review, see, e.g., Strohl, 2015. BioDrugs. 29(4):215-239.

A further object of the present invention is the isolated peptide targeting the MPER subdomain of the Spike protein described above, for use in preventing or inhibiting an infection by a virus of the Orthocoronavirinae subfamily, as defined hereinabove.

A further object of the present invention is a method of preventing or inhibiting an infection by a virus of the Orthocoronavirinae subfamily, as defined hereinabove, in a subject in need thereof, comprising administering to said subject the isolated peptide targeting the MPER subdomain of the Spike protein described above.

In one embodiment, the virus of the Orthocoronavirinae subfamily is a virus of the betacoronavirus genus, as defined hereinabove. In one embodiment, the virus of the Orthocoronavirinae subfamily is a virus of the betacoronavirus genus, and severe acute respiratory syndrome-related coronavirus species, as defined hereinabove. In one embodiment, the virus of the Orthocoronavirinae subfamily is a virus of the betacoronavirus genus, severe acute respiratory syndrome-related coronavirus species and strain SARS-CoV-2, as defined hereinabove. In one embodiment, the virus of the Orthocoronavirinae subfamily is SARS-CoV-2, as defined hereinabove.

In one embodiment, preventing or inhibiting the infection comprises blocking or hindering the fusion of the virus membrane with the subject's cell membrane.

### Examples

The present invention is further illustrated by the following examples.

### Materials and Methods

### Expression Vectors and Molecular Cloning

Expression system to sort membrane proteins on extracellular vesicles (EVs) was developed by CILOA SAS (granted patents EP 2 268 816 and U.S. Pat. No. 9,546,371; De Gassart et al., 2009. Cell Biol Int. 33(1):36-48).

### DNA^{S-EV}

Briefly, the S gene from SARS-CoV-2 Wuhan-Hu-1 isolate (Wu et al., 2020. Nature. 579(7798):265-269; GenBank accession number MN908947, version 3 of Mar. 18, 2020) (SEQ ID NO: 1), encoding the Spike protein (SEQ ID NO: 2), was codon-optimized for human cells, and further modified as follows:
substitution of the native Spike signal peptide (nucleotides 1 to 36 of SEQ ID NO: 1, corresponding to amino acids 1 to 12 of SEQ ID NO: 2) by the signal peptide of the metabotropic glutamate receptor 5 (mGluR5) (nucleic acid sequence with SEQ ID NO: 3, corresponding to the amino acid sequence with SEQ ID NO: 4);
two consecutive proline substitutions at positions K986 and V987 (SEQ ID NO: 2 numbering); and
a K776A substitution and a Q779G substitution (SEQ ID NO: 2 numbering).

-mGluR5
SEQ ID NO: 3
ATGGTGCTGCTGCTGATCCTGTCTGTGCTGCTCCTGAAAGAAGATGTGC
GGGGCTCTGCCCAGAGCACC
-mGluR5 encoded by SEQ ID NO: 3
SEQ ID NO: 4
MVLLLILSVLLLKEDVRGSAQST

This modified S gene (with SEQ ID NO: 5, encoding a modified Spike protein with SEQ ID NO: 6) was cloned in PacI-NotI in an in-house pCilA-DCTM eukaryotic expression vector, in order to generate a “DNA^{S-EV}” vector. pCilA-DCTM comprises a cytomegalovirus-human T-lymphotropic virus type I (CMV-HTLV-I) promoter upstream of the PacI-NotI expression cassette; and a bGH poly(A) and Ad2 VA1 sequence downstream of the PacI-NotI expression cassette.

This modified S gene was fused in C-terminus to a pilot peptide (herein named “CilPP”) which sorts the Spike protein to the surface of EVs (nucleic acid sequence with SEQ ID NO: 7, encoding CilPP with amino acid sequence SEQ ID NO: 8), through a 3-amino acid linker (nucleic acid sequence with SEQ ID NO: 9, encoding the linker with amino acid sequence SEQ ID NO: 10).

-Linker
SEQ ID NO: 9
TCTAGAGGC
-Linker encoded by SEQ ID NO: 9
SEQ ID NO: 10
SRG

### DNA^{S-Trim}

As was the case for the DNA^{S-EV} vector, the S gene from SARS-CoV-2 Wuhan-Hu-1 isolate (SEQ ID NO: 1) was codon-optimized for human cells, and further modified as follows:
substitution of the native Spike signal peptide (nucleotides 1 to 36 of SEQ ID NO: 1, corresponding to amino acid residues 1 to 12 of SEQ ID NO: 2) by the signal peptide of the metabotropic glutamate receptor 5 (mGluR5) (nucleic acid sequence with SEQ ID NO: 3, corresponding to the amino acid sequence with SEQ ID NO: 4);
two consecutive proline substitutions at positions K986 and V987 (SEQ ID NO: 2 numbering);
a K776A substitution and a Q779G substitution (SEQ ID NO: 2 numbering); and
truncation at amino acid residue 1208 (SEQ ID NO: 2 numbering).

This modified S gene (with SEQ ID NO: 11, encoding a modified Spike protein with SEQ ID NO: 12) was cloned in PacI-NotI in the in-house pCilA-DCTM eukaryotic expression vector, in order to generate a “DNA^{S-Trim}” vector.

This modified S gene was fused in C-terminus to a sequence comprising (i) a TEV cleavage site, (ii) a bacteriophage T4 foldon trimerization motif, and (iii) a 6×His tag (nucleic acid sequence with SEQ ID NO: 13, encoding the linker with amino acid sequence SEQ ID NO: 14).

### Pseudovirus Neutralization Assay

To establish pseudovirus neutralization assay, the following constructs were obtained. The SARS-CoV-2 S gene from plasmid pUC57-2019-nCoV-S-Hu (GenScript, Piscataway, USA) was truncated in its cytoplasmic tail of 19 amino acids in order to enhance pseudotyping efficiency and then subcloned into in-house expression vector (Johnson et al., 2020. J Virol. JVI.01062-20).

In the pNL4-3.Luc.R-E-luciferase reporter vector (NIH-AIDS Reagent Program, catalog number 3418), luciferase was replaced by Nano Luciferase (named hereafter “pNL4-3.NanoLuc”) (Connor et al., 1995. Virology. 206(2):935-944). Homo sapiens angiotensin I converting enzyme 2 (ACE2) gene (NM_021804.2, with SEQ ID NO: 15) and Homo sapiens transmembrane serine protease 2 (TMPRSS2) gene (NM_005656.4, with SEQ ID NO: 16) were both cloned into pcDNA3.1(+) from GenScript (Piscataway, USA).

-hACE2
SEQ ID NO: 15
ATGTCAAGCTCTTCCTGGCTCCTTCTCAGCCTTGTTGCTGTAACTGCTGCTCAGTC
CACCATTGAGGAACAGGCCAAGACATTTTTGGACAAGTTTAACCACGAAGCCGA
AGACCTGTTCTATCAAAGTTCACTTGCTTCTTGGAATTATAACACCAATATTACTG
AAGAGAATGTCCAAAACATGAATAATGCTGGGGACAAATGGTCTGCCTTTTTAA
AGGAACAGTCCACACTTGCCCAAATGTATCCACTACAAGAAATTCAGAATCTCA
CAGTCAAGCTTCAGCTGCAGGCTCTTCAGCAAAATGGGTCTTCAGTGCTCTCAGA
AGACAAGAGCAAACGGTTGAACACAATTCTAAATACAATGAGCACCATCTACAG
TACTGGAAAAGTTTGTAACCCAGATAATCCACAAGAATGCTTATTACTTGAACCA
GGTTTGAATGAAATAATGGCAAACAGTTTAGACTACAATGAGAGGCTCTGGGCT
TGGGAAAGCTGGAGATCTGAGGTCGGCAAGCAGCTGAGGCCATTATATGAAGAG
TATGTGGTCTTGAAAAATGAGATGGCAAGAGCAAATCATTATGAGGACTATGGG
GATTATTGGAGAGGAGACTATGAAGTAAATGGGGTAGATGGCTATGACTACAGC
CGCGGCCAGTTGATTGAAGATGTGGAACATACCTTTGAAGAGATTAAACCATTAT
ATGAACATCTTCATGCCTATGTGAGGGCAAAGTTGATGAATGCCTATCCTTCCTA
TATCAGTCCAATTGGATGCCTCCCTGCTCATTTGCTTGGTGATATGTGGGGTAGAT
TTTGGACAAATCTGTACTCTTTGACAGTTCCCTTTGGACAGAAACCAAACATAGA
TGTTACTGATGCAATGGTGGACCAGGCCTGGGATGCACAGAGAATATTCAAGGA
GGCCGAGAAGTTCTTTGTATCTGTTGGTCTTCCTAATATGACTCAAGGATTCTGG
GAAAATTCCATGCTAACGGACCCAGGAAATGTTCAGAAAGCAGTCTGCCATCCC
ACAGCTTGGGACCTGGGGAAGGGCGACTTCAGGATCCTTATGTGCACAAAGGTG
ACAATGGACGACTTCCTGACAGCTCATCATGAGATGGGGCATATCCAGTATGAT
ATGGCATATGCTGCACAACCTTTTCTGCTAAGAAATGGAGCTAATGAAGGATTCC
ATGAAGCTGTTGGGGAAATCATGTCACTTTCTGCAGCCACACCTAAGCATTTAAA
ATCCATTGGTCTTCTGTCACCCGATTTTCAAGAAGACAATGAAACAGAAATAAAC
TTCCTGCTCAAACAAGCACTCACGATTGTTGGGACTCTGCCATTTACTTACATGTT
AGAGAAGTGGAGGTGGATGGTCTTTAAAGGGGAAATTCCCAAAGACCAGTGGAT
GAAAAAGTGGTGGGAGATGAAGCGAGAGATAGTTGGGGTGGTGGAACCTGTGCC
CCATGATGAAACATACTGTGACCCCGCATCTCTGTTCCATGTTTCTAATGATTACT
CATTCATTCGATATTACACAAGGACCCTTTACCAATTCCAGTTTCAAGAAGCACT
TTGTCAAGCAGCTAAACATGAAGGCCCTCTGCACAAATGTGACATCTCAAACTCT
ACAGAAGCTGGACAGAAACTGTTCAATATGCTGAGGCTTGGAAAATCAGAACCC
TGGACCCTAGCATTGGAAAATGTTGTAGGAGCAAAGAACATGAATGTAAGGCCA
CTGCTCAACTACTTTGAGCCCTTATTTACCTGGCTGAAAGACCAGAACAAGAATT
CTTTTGTGGGATGGAGTACCGACTGGAGTCCATATGCAGACCAAAGCATCAAAG
TGAGGATAAGCCTAAAATCAGCTCTTGGAGATAAAGCATATGAATGGAACGACA
ATGAAATGTACCTGTTCCGATCATCTGTTGCATATGCTATGAGGCAGTACTTTTTA
AAAGTAAAAAATCAGATGATTCTTTTTGGGGAGGAGGATGTGCGAGTGGCTAAT
TTGAAACCAAGAATCTCCTTTAATTTCTTTGTCACTGCACCTAAAAATGTGTCTGA
TATCATTCCTAGAACTGAAGTTGAAAAGGCCATCAGGATGTCCCGGAGCCGTATC
AATGATGCTTTCCGTCTGAATGACAACAGCCTAGAGTTTCTGGGGATACAGCCAA
CACTTGGACCTCCTAACCAGCCCCCTGTTTCCATATGGCTGATTGTTTTTGGAGTT
GTGATGGGAGTGATAGTGGTTGGCATTGTCATCCTGATCTTCACTGGGATCAGAG
ATCGGAAGAAGAAAAATAAAGCAAGAAGTGGAGAAAATCCTTATGCCTCCATCG
ATATTAGCAAAGGAGAAAATAATCCAGGATTCCAAAACACTGATGATGTTCAGA
CCTCCTTTTAG
-hTMPRSS2
SEQ ID NO: 16
ATGGCTTTGAACTCAGGGTCACCACCAGCTATTGGACCTTACTATGAAAACCATG
GATACCAACCGGAAAACCCCTATCCCGCACAGCCCACTGTGGTCCCCACTGTCTA
CGAGGTGCATCCGGCTCAGTACTACCCGTCCCCCGTGCCCCAGTACGCCCCGAGG
GTCCTGACGCAGGCTTCCAACCCCGTCGTCTGCACGCAGCCCAAATCCCCATCCG
GGACAGTGTGCACCTCAAAGACTAAGAAAGCACTGTGCATCACCTTGACCCTGG
GGACCTTCCTCGTGGGAGCTGCGCTGGCCGCTGGCCTACTCTGGAAGTTCATGGG
CAGCAAGTGCTCCAACTCTGGGATAGAGTGCGACTCCTCAGGTACCTGCATCAAC
CCCTCTAACTGGTGTGATGGCGTGTCACACTGCCCCGGCGGGGAGGACGAGAAT
CGGTGTGTTCGCCTCTACGGACCAAACTTCATCCTTCAGGTGTACTCATCTCAGA
GGAAGTCCTGGCACCCTGTGTGCCAAGACGACTGGAACGAGAACTACGGGCGGG
CGGCCTGCAGGGACATGGGCTATAAGAATAATTTTTACTCTAGCCAAGGAATAG
TGGATGACAGCGGATCCACCAGCTTTATGAAACTGAACACAAGTGCCGGCAATG
TCGATATCTATAAAAAACTGTACCACAGTGATGCCTGTTCTTCAAAAGCAGTGGT
TTCTTTACGCTGTATAGCCTGCGGGGTCAACTTGAACTCAAGCCGCCAGAGCAGG
ATTGTGGGCGGCGAGAGCGCGCTCCCGGGGGCCTGGCCCTGGCAGGTCAGCCTG
CACGTCCAGAACGTCCACGTGTGCGGAGGCTCCATCATCACCCCCGAGTGGATC
GTGACAGCCGCCCACTGCGTGGAAAAACCTCTTAACAATCCATGGCATTGGACG
GCATTTGCGGGGATTTTGAGACAATCTTTCATGTTCTATGGAGCCGGATACCAAG
TAGAAAAAGTGATTTCTCATCCAAATTATGACTCCAAGACCAAGAACAATGACA
TTGCGCTGATGAAGCTGCAGAAGCCTCTGACTTTCAACGACCTAGTGAAACCAGT
GTGTCTGCCCAACCCAGGCATGATGCTGCAGCCAGAACAGCTCTGCTGGATTTCC
GGGTGGGGGGCCACCGAGGAGAAAGGGAAGACCTCAGAAGTGCTGAACGCTGC
CAAGGTGCTTCTCATTGAGACACAGAGATGCAACAGCAGATATGTCTATGACAA
CCTGATCACACCAGCCATGATCTGTGCCGGCTTCCTGCAGGGGAACGTCGATTCT
TGCCAGGGTGACAGTGGAGGGCCTCTGGTCACTTCGAAGAACAATATCTGGTGG
CTGATAGGGGATACAAGCTGGGGTTCTGGCTGTGCCAAAGCTTACAGACCAGGA
GTGTACGGGAATGTGATGGTATTCACGGACTGGATTTATCGACAAATGAGGGCA
GACGGCTAA

### Production and Purification of S Trimer (S-Trim)

The production and purification of coronavirus S trimer was previously described by Pallesen et al. (2017. Proc Natl Acad Sci USA. 114(35):E7348-E7357).

In brief, HEK293T cells were transfected with DNA^{S-Trim} plasmid using polyethylenimine (PEI) and harvested 48 h later. The cell pellet was then resuspended in 1×PBS supplemented with 0.5 mM PMSF and protease inhibitors (Protease Inhibitor Cocktail set III, EDTA-free, Calbiochem).

Lysis buffer (1× PBS, 2% octyl β-D-1 thioglucopyranoside, 12 mM MgCl₂, 0.5 mM PMSF, protease inhibitors and DNase I) was then added and incubated for 30 minutes at 4° C.

The lysate was clarified by centrifugation at 800 rpm for 10 minutes at 4° C., then at 15 000 rpm for 15 minutes at 4° C. The supernatant was collected, equilibrated with 10 mM imidazole, and loaded onto IMAC resin (1 mL HisTALON™ Superflow Cartridge [Takara] coupled to a fast protein liquid chromatography system Äkta FPLC UPC-900/P-920 [GE Healthcare]).

S-Trim was eluted with 150 mM imidazole. Fractions containing pure S-Trim were identified by Western Blot and pooled. Protein concentration was estimated with bicinchoninic acid (BCA) assay (Pierce BCA Protein Assay Kit, ThermoFisher Scientific), by measuring absorbance at 562 nm.

### Production of EVs Harboring Spike Protein (S-EVs) in Mammalian Cells

EVs were produced in HEK293T cells obtained from American Type Culture Collection (ATCC). Cells were cultured in DMEM supplemented with 5% heat-inactivated fetal bovine serum (iFBS), 2 mM GlutaMAX and 5 μg/mL gentamicin at 37° C. in a 5% CO₂ humidified incubator. HEK293T cells were routinely tested and found negative by MycoAlert™ mycoplasma detection kit (Lonza Nottingham, Ltd.).

DNA^{S-EV} were transfected into HEK293T cells using PEI. In order to generate large-scale exosome production, HEK293T cells were plated into cell chambers of 10 trays in 1 L of complete medium. Twenty-four hours post-transfection, cultures were fed with medium supplemented with EV-free iFBS and incubated for a further 48 hours.

### S-EVs Purification

Cell culture medium was harvested from transiently transfected HEK293T cells and S-EV isolation was performed as previously described (Taylor & Shah, 2015. Methods. 87:3-10; Desplantes et al., 2017. Sci Rep. 7(1):1032). Briefly, cell culture supernatant was clarified by two consecutive centrifugations: 10 minutes at 1300 rpm and 15 minutes at 4000 rpm, both at 4° C., followed by filtration through 0.22 μm membrane filters.

The supernatant was then concentrated by ultra-filtration and diafiltration and load onto size exclusion chromatography (SEC) columns (Sephacryl S1000, GE Healthcare). Fractions containing EVs biomarkers (CD81 and CD63) were identified by ELISA. EV fractions containing Spike protein identified by Western-blot were pooled, concentrated when necessary, and used for analysis and injections.

### ExoView EVs Characterization

Purified S-EVs were sized and characterized using the Single Particle Interferometric Reflectance Imaging Sensor (SP-IRIS) technology using the ExoView R100 platform (NanoView Biosciences, Boston, USA).

Tetraspanin kit chips (EV-TETRA-C), with capture antibodies against CD81, CD63, CD9 and IgG (MIgG), were used to detect S-EVs.

S-EVs at 10¹⁰ EVs/mL were coated on chips, then probed with detection antibodies provided with the kit (CD81/CF55, CD63/CF647, CD9/CF488A) according to the manufacturer's protocol. The chips were imaged with ExoView R100, label-free counts were used for S-EV size determination (with a size range set from 50 to 200 nm).

### SDS-PAGE, Western Blotting and Antibodies

Protein concentration of S-EVs was measured using the BCA assay (Pierce BCA Protein Assay Kit, ThermoFisher Scientific).

S-EVs or S-Trim preparations were separated by SDS-PAGE on a 4-15% acrylamide gel (4-15% Mini-PROTEAN® TGX Stain-Free™ Gel, Bio-Rad) and subsequently transferred onto PVDF membrane. For Western blotting in non-reducing conditions, a loading buffer without DTT was used.

Immunodetection of Spike protein was carried out with primary antibodies against either the S1 head subunit (anti-SARS-CoV-2 Spike S1, rabbit monoclonal antibody [HL6], GTX635654, Genetex) or the S2 stem subunit (anti-SARS-CoV-2 Spike S2, mouse monoclonal antibody [1A9], GTX632604, Genetex) or anti-CilPP (in-house antibody raised in rabbit).

Membranes were then incubated with the corresponding secondary HRP-conjugated antibodies (donkey anti-mouse or anti-rabbit HRP, #715-035-150 or #711-035-152, Jackson ImmunoResearch). The signals were detected using an enhanced chemiluminescence detection kit (Super Signal West Pico Plus, 34580, ThermoFischer Scientific) and membranes imaged with ChemiDoc Imaging System (BioRad).

The same anti-S1 and anti-S2 antibodies, as well secondary antibodies, were also used to detect the Spike protein on S-EVs by ELISA (see below).

### Ethics Statement

All animal studies were performed in accordance with national regulations and approved prior to experimentation by the Ethics Committee for Animal Testing Languedoc-Roussillon (national agreement CEEA-036) and French Ministry of Research with the reference APAFIS #24869 (Apr. 10, 2020). All the procedures and protocols were performed at DMEM unit of INRAE-UM in Montpellier, France (Veterinary Services National Agreement no E34-172-10, Mar. 4, 2019).

### Mice and Study Design

Female BALB/cAnNCrl mice, 6 weeks old, were purchased from Charles River (Italy) and placed into four groups of n=6. Mice were housed in individually ventilated cages with controlled environmental parameters: 24° C., 12 hours/12 hours light/dark cycle, nesting cotton squares for enrichment, free access to standard A04 irradiated food (SAFE, R04-25) and tap water. Behavior and health status were observed daily and weight checked weekly. One week after their arrival, animals received 2 prime immunizations at day 0 and 21 and a boost immunization at day 42.
group DNA^{S-EV}/S-EV:
   prime immunization: DNA^{S-EV} vectors were injected using a Gene Gun (BioRad, Helios) into the abdomen of mice previously shaved. Each mouse received 3 cartridges coated with gold beads containing DNA^{S-EV} corresponding to a total of 3 μg of DNA, at day 0 and 21;
   boost immunization: 10 μg of S-EVs were injected subcutaneously in each mouse, in 100 μL volume of PBS, at day 42.
group DNA^{S-EV}/S-Trim:
   prime immunization: DNA^{S-EV} vectors were injected using a Gene Gun (BioRad, Helios) into the abdomen of mice previously shaved. Each mouse received 3 cartridges coated with gold beads containing DNA^{S-EV} corresponding to a total of 3 μg of DNA, at day 0 and 21;
   boost immunization: S-Trim was injected subcutaneously in each mouse, in 100 μL volume of PBS, at day 42.
group S-EV/S-EV:
   prime immunization: 10 μg of S-EVs were injected subcutaneously in each mouse, in 100 μL volume of PBS, at day 0 and 21;
   boost immunization: 10 μg of S-EVs were injected subcutaneously in each mouse, in 100 μL volume of PBS, at day 42.
group PBS: negative control, received PBS injections at days 0, 21 and 42.

Sera were collected via submandibular bleeding before each immunization: 70 to 100 mL of blood was collected with GOLDENROD animal Lancets (3 mm, Genobios). All animals were euthanized at day 63 and spleens were collected for cellular response analysis.

### S1/S2 and Antigen Specific IgG ELISA

SARS-CoV-2 specific antibody titers of sera were determined by ELISA. Briefly, MaxiSorp ELISA plates (Nunc) were 2 μg/mL of SARS-CoV-2 Spike overlapping peptide pools corresponding to S1 or S2 subunits (PepMix™ SARS-CoV-2 Spike glycoprotein, JPT, #PM-WCPV-S) in 100 μL, 50 mM sodium carbonate/bicarbonate pH 9.6 buffer per well, overnight at 4° C.

Coated plates were washed 3 times with 200 μL of 1×PBS and saturated for 1 hour at 37° C. with 200 μL 3% BSA in 1×PBS per well.

Plates were washed three times with 1×PBS, then incubated in 3% BSA and 5% FBS with 3-fold mouse sera dilutions (starting from 1:100) for 2 hours at 37° C. This was followed by 3 washes with 200 μL of 1×PBS per well and incubation with 100 μL per well of secondary donkey anti-mouse HRP conjugated antibody diluted 1:10000 in 3% BSA in 1×PBS.

Following incubation with the secondary antibody, plates were washed 5 times with 200 μL of 1×PBS per well and developed with 100 μL of TMB per well (Bio-Rad #R8/R9) for 30 minutes. The reaction was stopped by adding 50 μL of stop solution (2 N sulfuric acid) per well.

The 450 nm-absorbance was read using ClarioStar Plus plate reader (BMG Labtech). The reciprocal endpoint titers were defined as the dilution with the 450 nm OD 3 times higher than the background. Undetectable antibody titers (negative values) were assigned values of 0.

ELISA analysis of Spike protein exposure on EVs was performed as above, with few modifications. MaxiSorp ELISA plates were coated with S-EVs at 10 μg/mL in 100 μL 50 mM sodium carbonate/bicarbonate pH 9.6 buffer per well, overnight at 4° C. Saturation was performed with 3% BSA in 1×PBS and followed by incubation with dilutions of either anti-S1 or S2 antibodies. Secondary HRP-conjugated donkey anti-rabbit and anti-mouse antibodies were used, respectively.

### IFN-γ ELISpot Assay

Single cell suspensions were prepared from spleens of mice euthanized at day 63. Splenocytes from all immunized mice were analyzed in to pools of three animals. Total T-cells were isolated (EasySep™ Mouse T Cell Isolation Kit, StemCells #19851), and 2×10⁵ T-cells were stimulated for 18 hours in cell culture medium (RPMI 1640 with L-glutamine, 25 mM Hepes, 10% FBS and 5 μg/mL gentamycin) at 37° C. and 5% CO₂ with overlapping peptide pools representative of either S1 or S2 subunit of the Spike protein of SARS-CoV-2 (PepMix™ SARS-CoV-2 Spike glycoprotein, JPT, #PM-WCPV-S) at 1 μg/mL each, or DMSO in 96-well ELISpot IFN-γ plates (Mabtech #3321-4APW-2) in triplicates. As positive control stimuli, 0.5-1×10⁵ cells were stimulated with PHA-L (eBioscience, #15556286) at 1.25-5 μg/mL.

Following the 18-hour incubation, plates were treated according to the manufacturer's protocol. Acquisition and analysis were performed with a CTL Immunospot S6 analyzer.

Measurement values from DMSO-stimulated cells were subtracted from all the measurements. Negative values were corrected to 0. Results are represented as a number of spot forming cells (SPC) per 1×10⁶ T-cells.

### Pseudovirus Neutralization Assay

To produce SARS-CoV-2 pseudoviruses, SARS-CoV-2 truncated Spike expression and lentiviral pNL4-3.NanoLuc vectors were co-transfected into HEK293T cells using PEI.

Pseudovirus-containing supernatant was collected 48 hours after, filtered through 0.45-μm filters, aliquoted and stored at −80° C.

To perform the neutralization assay, 2-fold mouse sera dilutions (starting from 1:10) collected at day 63 were mixed with equal volumes of pre-tittered (1000 times the background of fluorescence) Spike-pseudotyped HIV-NanoLuc viruses (1:2), incubated for 1 hour at 37° C., 5% CO₂ and then added to HEK293T cells transiently expressing ACE2 receptor and TMPRSS2 protease in 96-well plates (in triplicates) for 1 hour at 37° C., 5% CO₂. Cell culture medium was then changed. After a 48-hour incubation, cells were lysed and luciferase activity (RLU, relative light units) was measured using the Bright-Glo™ Luciferase Assay System (Promega). Background luminescence produced by cells-only controls (no pseudotyped virus) and positive luminescence produced by pseudotyped virus-infected cells were included and served to determine percentage of neutralization as 100% and 0%, respectively. Neutralization titers were defined as the sera dilutions that neutralize 50% of the virus.

### Protein Sequence Alignment

Multiple sequence alignment was performed with Clustal Omega tool (EMBL-EBI).

### Results

Predicted B- and T-Cell Epitope Homology within the Spike Protein of SARS-CoV-2, SARS-CoV, Common Cold Viruses, HIV Gp41 and CoVEVax Vaccine Candidates

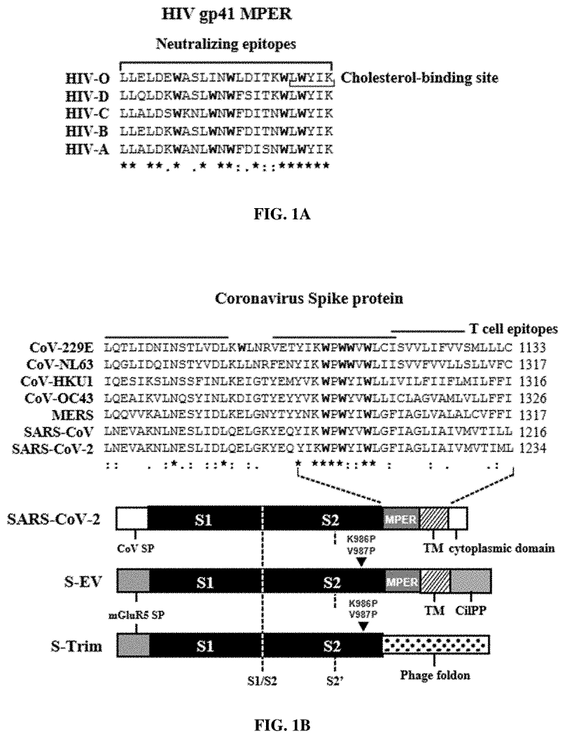

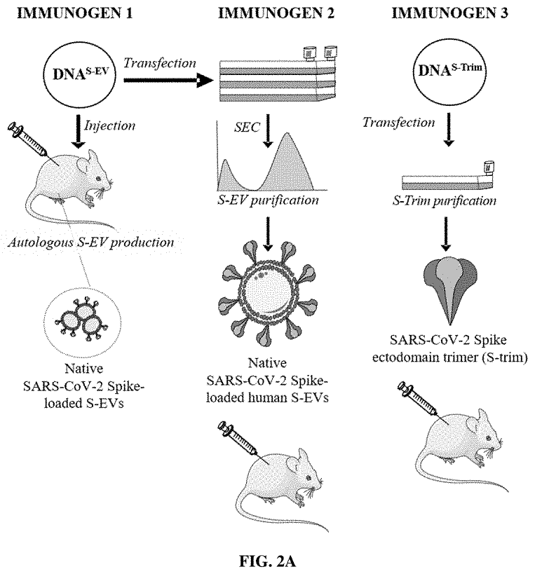

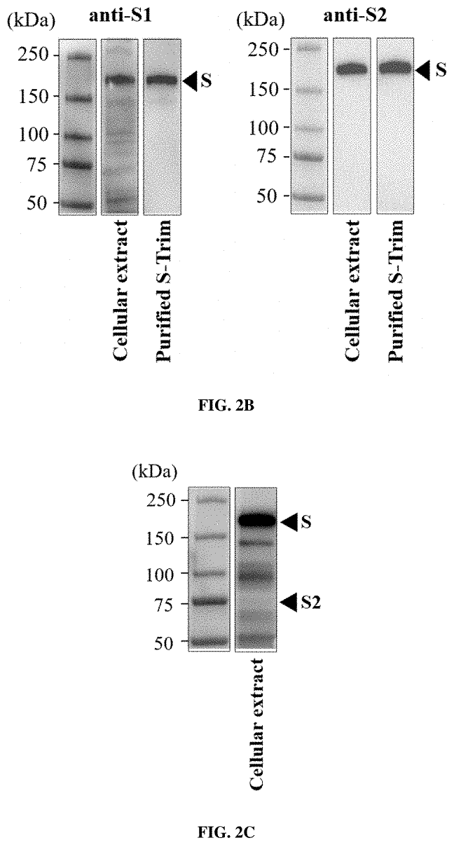

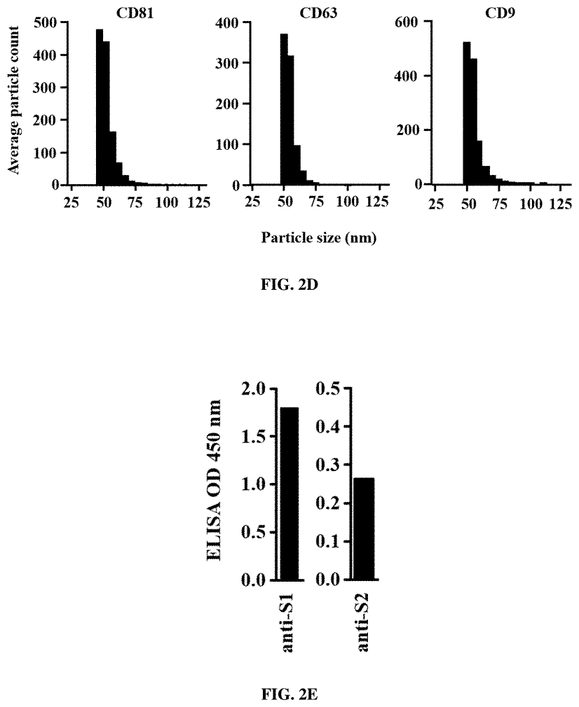

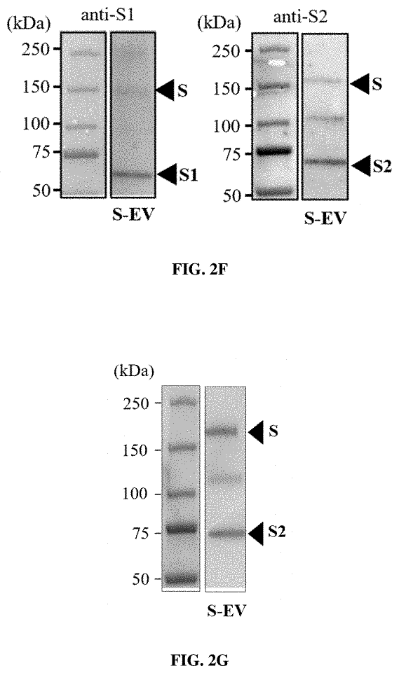

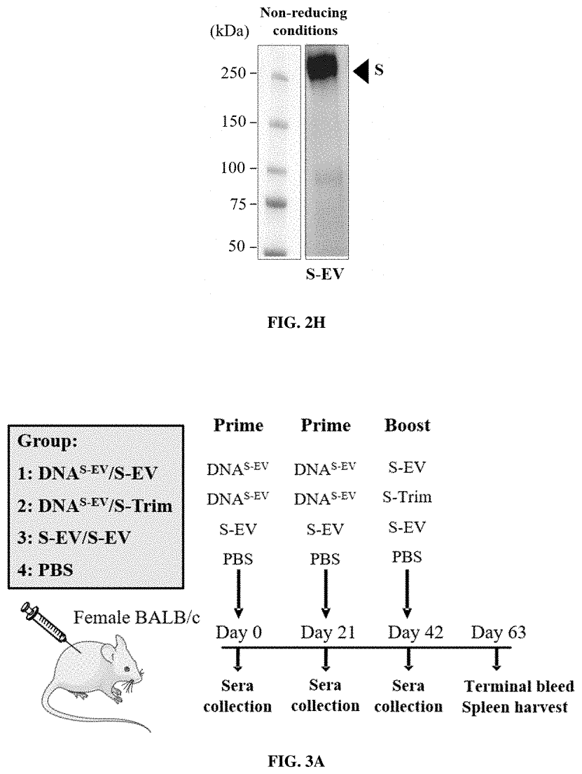

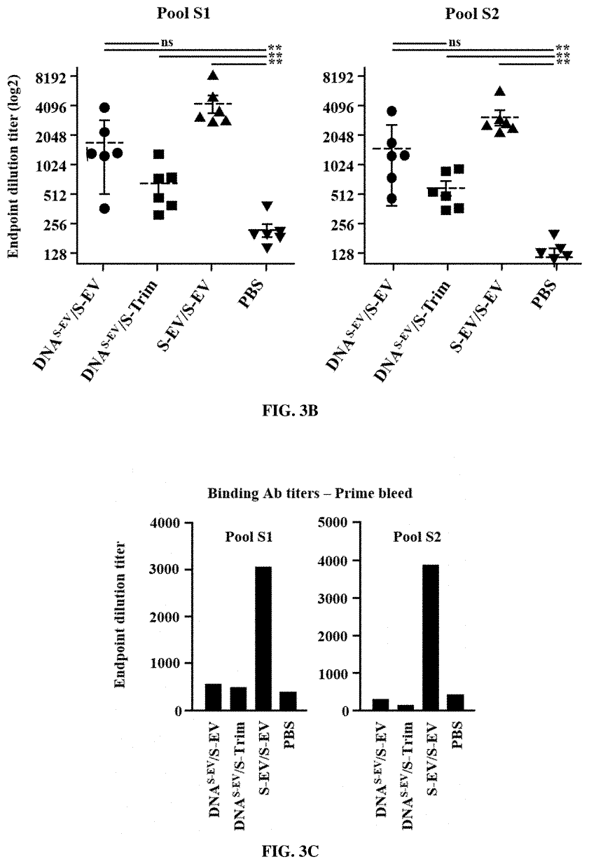

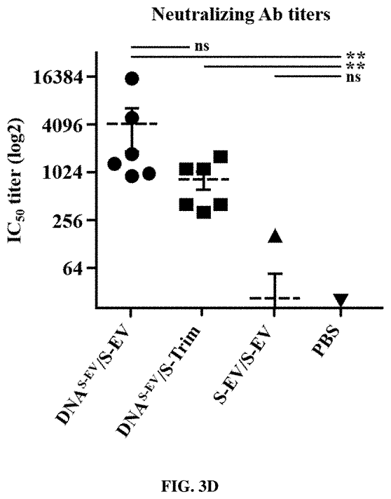

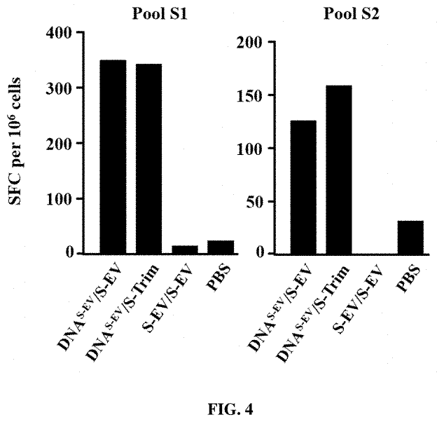

For optimal vaccine design, several envelope protein sequences were analyzed, considering B and T epitopes not only from coronaviruses but also from HIV strains (FIG. 1).

The HIV's MPER sub-region required for the membrane fusion of HIV's protein TM contains a tryptophan-rich cluster, a cholesterol-binding domain and a neutralizing epitope conserved through all HIV clades (FIG. 1A) (Liu et al., 2018. Protein Cell. 9(7):596-615).

Interestingly, the S2 MPER sub-region of all coronaviruses (four human “common cold” coronaviruses, MERS, SARS-CoV and SARS-CoV-2) possesses an analogous highly-conserved tryptophan cluster and a putative cholesterol-binding region (FIG. 1B, top).

Similarly to HIV, this conserved MPER sub-region would contain a neutralizing B epitope and includes one of the T-cell epitopes cross-reactive in unexposed individuals (Mateus et al., 2020. Science. eabd3871). In addition, the most important reactive T-cell epitope of SARS-CoV, entirely conserved in SARS-CoV-2, is located in the transmembrane domain (FIG. 1B, top) (Wang et al., 2004. J Virol. 78(11):5612-5618). Altogether, these facts demonstrate the importance of including the MPER subregion and transmembrane domain in a vaccine.

The first molecular construct (S-Trim) was often used in other vaccines; it comprises an immunogen containing the full S1 subunit, the S2 subunit truncated at Q1219 (SEQ ID NO: 6 numbering; or residue 1208 with SEQ ID NO: 2 numbering) and an additional phage Foldon which allows artificial trimerization. This S1/S2 immunogen is soluble because it is devoid of the MPER subregion and transmembrane domains (FIG. 1B, bottom).

The second construct (S-EV) is a Spike protein, comprising subunits S1 and S2, anchored in a natural membrane. It contains the MPER subregion and transmembrane domain, identified as potentially important in triggering neutralizing humoral and cellular immune responses and a function in membrane anchoring (FIG. 1B, bottom).

### Production and Characterization of the CoVEVax Vaccine Candidates

It is well-known that T-cell immune responses can be elicited by DNA vaccines, but that a protein boost is required for high humoral responses.

Thus, we chose to develop a two-component vaccine, the first one being the DNA^{S-EV} leading to in situ production and loading of Spike protein S1 and S2 subunits on autologous EVs (S-EVs), and the second one being exogenously produced and purified S-EVs (FIG. 2A).

As a control, we produced soluble S-Trim as a third immunogen (FIG. 2A). The expression of S-Trim was analyzed by Western blotting. A band of the size expected for a non-mature protein was revealed when probed with either anti-S1 or anti-S2 antibody (FIG. 2B), indicating that the artificial S-Trim was correctly expressed but remained immature and was not processed into S1 and S2.

DNA^{S-EV}, when transfected in HEK293T cells, allowed the expression of two main proteins detected by an anti-CilPP antibody, the first with a size expected for an immature S and the second with a size expected for a mature S2 subunit (FIG. 2C). This partial maturation of envelope proteins in cells was previously described for coronaviruses and HIV (Park et al., 2016. Proc Natl Acad Sci USA. 113(43):12262-12267; Moulard et al., 1999. Virus Res. 60(1):55-65).

Secreted EVs were purified and analyzed for their size and protein content. Using the ExoView platform, we observed that S-EVs are specifically captured by anti-CD81, anti-CD63 and anti-CD9 antibodies and exhibit a size of ˜55 nm, all being signatures of EVs (FIG. 2D). ELISA analysis revealed that these purified S-EVs harbor both S1 and S2 subunits at their surface (FIG. 2E). Interestingly, when analyzed by Western blot, the secreted EVs contained S immature protein, but predominantly, mature S1 and S2 subunits (FIGS. 2F and 2G); all being associated as a natural S1/S2 trimer, naturally anchored in the EV membrane (FIG. 2H).

Humoral Response in Mice Immunized with CoVEVax

Mice were immunized at day 0, 21 and 42 with 3 different combinations of CoVEVax immunogens, or with PBS as a negative control (FIG. 3A). Notably, in this study, no adjuvants were administered (DNA^{S-EV}, purified S-EVs and soluble S-Trim were suspended in PBS). Sera were analyzed at day 42 (prime bleed) and day 63 (terminal bleed) together with spleen collection (FIG. 3A). The humoral immune response induced by CoVEVax was assessed by ELISA. To determine SARS-CoV-2 specific titers, we coated ELISA plates with peptide pools representative of either the S1 or S2 subunit.

Compared to the PBS group, all immunizations revealed evidence of antibodies directed against both S1 and S2 subunits after the protein boost. The mean titers against the S1 subunit obtained with sera of group 1 (DNA^{S-EV}/S-EV), group 2 (DNA^{S-EV}/S-Trim) and group 3 (S-EV/S-EV) were 1716, 663 and 4316, respectively. The mean titers against the S2 subunit obtained with sera of group 1, group 2 and group 3 were 1494, 591, 3116, respectively (FIG. 3B).

The lowest titers were obtained with S-Trim boost injection and no S-EV injection; average titers were obtained with one S-EV boost injection; the highest titers resulted from three S-EV injections. Hence, titers seem to correlate with the number of S-EV injections, which are important to mount an antibody response. Accordingly, sera from day 42 showed elevated ELISA titers within group 3 only (FIG. 3C).

Surprisingly, the humoral response against both S1 and S2 corresponding peptide pools was of a similar level, even though the S1 “head” subunit, with its receptor binding domain (RBD), is widely recognized as the main immunogen; here, when presented on EVs, the S2 “stem” subunit acts as a potent antigen as well.

In the depicted ELISA experiments, only the humoral response against linear epitopes was measured. We hypothesized that the Spike protein, when presented on EVs, would also trigger an immune response against conformational epitopes that are exhibited by the virus, and which are, generally, the true targets of neutralizing antibodies. To this end, the neutralizing activity of the sera was quantified with a commonly used pseudotyped lentivirus neutralization assay (FIG. 3D).

In brief, serial dilutions of sera were mixed with Spike-pseudotyped viruses and then used to infect HEK293T cells expressing human ACE2 receptor and TMPRSS2 protease. The results showed that, although group 3 displayed binding the highest ELISA titers, only one animal developed a low level (mean titer=160) of neutralizing antibodies, while the rest of the group displayed none, similarly to the negative control group. In contrast, mice in group 2 have produced a good level of neutralizing antibodies, with a mean titer of 827 (range: 320-1600), i.e., similar to the mean neutralizing titers reported for other vaccines (Folegatti et al., 2020. Lancet. 396(10249):467-478; Jackson et al., 2020. N Engl J Med. NEJMoa2022483; Yang et al., 2020. Nature. 10.1038/s41586-020-2599-8).

Most surprisingly, the sera of mice from group 1 exhibited very high level of neutralizing antibodies (mean titer=4173; range: 900-15300) (FIG. 3D).

Cellular Immune Response in Mice Immunized with CoVEVax

Finally, we looked at the T-cell mediated immunity, a second type of protective immune response, using ELISpot assay measuring antigen specific IFN-γ production.

As before, we aimed to identify and discriminate between the immune responses against S1 and S2 subunits. Mice immunized with S-EVs only (group 3) did not develop cellular immune response as measured by IFN-γ ELISpot.

By contrast, the two groups (1 and 2) immunized by two DNA^{S-EV} primes and one protein boost (S-EV or S-Trim) developed strong cellular immune response, reaching ˜350 SFC and ˜150 SFC per 10⁶ T-cells for S1 and S2 immunogens, respectively (FIG. 4).

### DISCUSSION

In conclusion, we have developed a unique EV-based vaccine candidate harboring a fully native and mature Spike protein from SARS-CoV-2, that triggers both a strong neutralizing response and a strong cellular immune response.

These responses were obtained in the absence of any other viral component and without adjuvants. They are directed against both S1 “head” and S2 “stem” subunits of the Spike protein, which is important for a broad coverage of the vaccine in regard to the high conservation of the S2 subunit in all coronavirus clades.

The two components of the CoVEVax are crucial to obtain both types of protective immune responses, the prime DNA^{S-EV}-based immunization triggering T-cell response, and the boost protein-based immunization (either with S-EV or S-Trim) rising the humoral response.

The observed strong neutralizing activity prompt us to expect that CoVEVax is unlikely to promote the induction of antibodies which can trigger antibody dependent enhancement (ADE) adverse events.

Additionally, this EV-based vaccine, which contains highly conserved sequences, will in all likelihood exhibit protection against antigenic drift that began to appear for SARS-CoV-2 (Korber et al., 2020. Cell. 182(4):812-827; Becerra-Flores & Cardozo, 2020. Int J Clin Pract. e13525).

1. A nucleic acid comprising:
(i) a sequence of a S gene coding for a Spike protein from a virus of the Orthocoronavirinae subfamily, or a variant thereof; and
(ii) a sequence coding for a pilot peptide which interacts with ESCRT proteins.

2. The nucleic acid according to claim 1, wherein the virus of the Orthocoronavirinae subfamily is Severe Acute Respiratory Syndrome Coronavirus 2 (SARS-CoV-2).

3. The nucleic acid according to claim 1, wherein the S gene has a nucleic acid sequence coding for a Spike protein with SEQ ID NO: 2, or a variant thereof.

4. The nucleic acid according to claim 1, wherein the S gene has a nucleic acid sequence coding for a variant of the Spike protein with SEQ ID NO: 6.

5. The nucleic acid according to claim 1, wherein the pilot peptide comprises at least one YxxL motif with SEQ ID NO: 17 or DYxxL motif with SEQ ID NO: 20, and at least one PxxP motif with SEQ ID NO: 24, in which “x” represents any amino acid residue.

6. The nucleic acid according to claim 1, wherein the pilot peptide comprises an amino acid sequence with SEQ ID NO: 8 or a variant thereof, with the proviso that a variant of SEQ ID NO: 8 retains three YxxL motifs with SEQ ID NO: 17 and four PxxP motifs with SEQ ID NO: 24, in which “x” represents any amino acid residue.

7. The nucleic acid according to claim 1, being inserted into a nucleic acid expression vector, and being operably linked to regulatory elements.

8. An extracellular vesicle harboring at its external surface a Spike protein from a virus of the Orthocoronavirinae subfamily “S-EV”, or a variant thereof.

9. The extracellular vesicle according to claim 8, wherein the virus of the Orthocoronavirinae subfamily is Severe Acute Respiratory Syndrome Coronavirus 2 (SARS-CoV-2)

10. The extracellular vesicle according to claim 8, wherein the extracellular vesicle is an exosome.

11. The extracellular vesicle according to claim 8, wherein the extracellular vesicle is an exosome having a diameter ranging from about 30 nm to about 120 nm.

12. The extracellular vesicle according to claim 8, being obtainable by a method comprising steps of:
1) transfecting cells with a nucleic acid comprising:
a. a sequence of a S gene coding for a Spike protein from a virus of the Orthocoronavirinae subfamily, or a variant thereof; and
b. a sequence coding for a pilot peptide which interacts with ESCRT proteins;
2) culturing the transfected cells for a time sufficient to allow extracellular vesicle production; and
3) purifying said extracellular vesicle.

13. A population of extracellular vesicles according to claim 8.

14. A method of immunizing a subject in need thereof against a virus of the Orthocoronavirinae subfamily, comprising the steps of:
1) at least one priming step, comprising administering to said subject a nucleic acid comprising:
a. a sequence of a S gene coding for a Spike protein from a virus of the Orthocoronavirinae subfamily, or a variant thereof; and
b. a sequence coding for a pilot peptide which interacts with ESCRT proteins; and
2) at least one boosting step, comprising:
a. administering to said subject an extracellular vesicle harboring at its external surface a Spike protein from a virus of the Orthocoronavirinae subfamily “S-EV”, or a variant thereof, or
b. administering to said subject a trimeric Spike protein from a virus of the Orthocoronavirinae subfamily,
thereby immunizing the subject against a virus of the Orthocoronavirinae subfamily.

15. The method of immunizing a subject according to claim 14, wherein the virus of the Orthocoronavirinae subfamily is Severe Acute Respiratory Syndrome Coronavirus 2 (SARS-CoV-2).

16. The method of immunizing a subject according to claim 14, wherein the period of time between the at least one priming step and the at least one boosting step ranges from about 2 weeks to about 1 month.

17. The method of immunizing a subject according to claim 14, wherein the method comprises two iterations of the priming step and one iteration of the boosting step.

18. The method of immunizing a subject according to claim 17, wherein the period of time between each iteration ranges from about 2 weeks to about 1 month.


SEQUENCE LISTING (56 sequences)

SEQ ID NO: 1 - DNA, 3822 bp, SARS-CoV-2 Wuhan-Hu-1 isolate
  S gene
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, SARS-CoV-2 Wuhan-Hu-1 isolate
  Spike protein encoded by SEQ ID NO 1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 69 bp, Artificial Sequence
  mGluR5
atggtgctgctgctgatcctgtctgtgctgctcctgaaagaagatgtgcggggctctgcccagagcacc

SEQ ID NO: 4 - protein, 23 aa, Artificial Sequence
  mGluR5
MVLLLILSVLLLKEDVRGSAQST

SEQ ID NO: 5 - DNA, 3735 bp, Artificial Sequence
  Variant of the S gene
atggtgctgctgctgatcctgtctgtgctgctcctgaaagaagatgtgcggggctctgcccagagcaccggtcaatgtgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgcgcctcttaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggatgccaataccggcgaagtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgctgccctgcagatcccctttgctatgcagatggcctacagattcaacggcatcggagtgacccagaatgtgctgtacgagaaccagaagctgatcgccaaccagttcaacagcgccatcggcaagatccaggacagcctgtctagcacagccagcgctctgggaaagctgcaggacgtggtcaaccagaatgcccaggcactgaacaccctggtcaagcagctgagcagcaatttcggcgccatcagctctgtgctgaacgatatcctgagcagactggaccctcctgaggccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaggaactggggaagtacgagcagtacatcaagtggccctggtacatctggctgggctttatcgccggactgattgccatcgtgatggtcacaatcatgctg

SEQ ID NO: 6 - protein, 1245 aa, Artificial Sequence
  Variant Spike protein encoded by SEQ ID NO 5
MVLLLILSVLLLKEDVRGSAQSTGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDANTGEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIML

SEQ ID NO: 7 - DNA, 138 bp, Artificial Sequence
  CilPP
gcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 8 - protein, 45 aa, Artificial Sequence
  CilPP encoded by SEQ ID NO 7
APHFPEISFPPKPDSDYQALLPSAPEIYSHLSPTKPDYINLRPAP

SEQ ID NO: 9 - DNA, 9 bp, Artificial Sequence
  Linker
tctagaggc

SEQ ID NO: 10 - protein, 3 aa, Artificial Sequence
  Linker encoded by SEQ ID NO 9
SRG

SEQ ID NO: 11 - DNA, 3657 bp, Artificial Sequence
  Modified S gene
atggtgctgctgctgatcctgtctgtgctgctcctgaaagaagatgtgcggggctctgcccagagcaccggtcaatgtgtgaacctgaccaccagaacacagctgcctccagcctacaccaacagcttcaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagctctggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctaccagccttacagagtggtggtgctgagcttcgagctgctgcatgctcctgccacagtgtgcggccctaagaaaagcaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgcgcctcttaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggatgccaataccggcgaagtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgctgccctgcagatcccctttgctatgcagatggcctacagattcaacggcatcggagtgacccagaatgtgctgtacgagaaccagaagctgatcgccaaccagttcaacagcgccatcggcaagatccaggacagcctgtctagcacagccagcgctctgggaaagctgcaggacgtggtcaaccagaatgcccaggcactgaacaccctggtcaagcagctgagcagcaatttcggcgccatcagctctgtgctgaacgatatcctgagcagactggaccctcctgaggccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaggaactggggaagtacgagcag

SEQ ID NO: 12 - protein, 1219 aa, Artificial Sequence
  Modified Spike protein encoded by SEQ ID NO 11
MVLLLILSVLLLKEDVRGSAQSTGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDANTGEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 13 - DNA, 153 bp, Artificial Sequence
  Trimerization foldon
agcggcagagagaacctgtacttccaaggcggaggcggcggaagcggctatattcctgaagctcctagagatggccaggcctacgtgcggaaagatggcgaatgggtcctgctgagcacctttctgggacaccaccaccatcaccattagtaa

SEQ ID NO: 14 - protein, 49 aa, Artificial Sequence
  Trimerization foldon encoded by SEQ ID NO 13
SGRENLYFQGGGGGSGYIPEAPRDGQAYVRKDGEWVLLSTFLGHHHHHH

SEQ ID NO: 15 - DNA, 2418 bp, Homo sapiens
  hACE2
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 16 - DNA, 1479 bp, Homo sapiens
  hTMPRSS2
atggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaa

SEQ ID NO: 17 - protein, 4 aa, Artificial Sequence
  Pilot peptide, YxxL motif
  SITE 2: Xaa represents any amino acid residue
  SITE 3: Xaa represents any amino acid residue
YXXL

SEQ ID NO: 18 - protein, 4 aa, Artificial Sequence
  Pilot peptide, YxxL motif
YINL

SEQ ID NO: 19 - protein, 4 aa, Artificial Sequence
  Pilot peptide, YxxL motif
YSHL

SEQ ID NO: 20 - protein, 5 aa, Artificial Sequence
  Pilot peptide, DYxxL motif
  SITE 3: Xaa represents any amino acid residue
  SITE 4: Xaa represents any amino acid residue
DYXXL

SEQ ID NO: 21 - protein, 5 aa, Artificial Sequence
  Pilot peptide, DYxxL motif
DYINL

SEQ ID NO: 22 - protein, 4 aa, Artificial Sequence
  Pilot peptide, YxxF motif
  SITE 2: Xaa represents any amino acid residue
  SITE 3: Xaa represents any amino acid residue
YXXF

SEQ ID NO: 23 - protein, 5 aa, Artificial Sequence
  Pilot peptide, DYxxF motif
  SITE 3: Xaa represents any amino acid residue
  SITE 4: Xaa represents any amino acid residue
DYXXF

SEQ ID NO: 24 - protein, 4 aa, Artificial Sequence
  Pilot peptide, PxxP motif
  SITE 2: Xaa represents any amino acid residue
  SITE 3: Xaa represents any amino acid residue
PXXP

SEQ ID NO: 25 - protein, 4 aa, Artificial Sequence
  Pilot peptide, PxxP motif
PSAP

SEQ ID NO: 26 - protein, 4 aa, Artificial Sequence
  Pilot peptide, PxxP motif
PTAP

SEQ ID NO: 27 - protein, 40 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 7: Xaa represents any amino acid residue
  SITE 8: Xaa represents any amino acid residue
  SITE 10: Xaa represents any amino acid residue
  SITE 11: Xaa represents any amino acid residue
  SITE 13: Xaa represents any amino acid residue
  SITE 15: Xaa represents any amino acid residue
  SITE 17: Xaa represents any amino acid residue
  SITE 18: Xaa represents any amino acid residue
  SITE 20: Xaa represents any amino acid residue
  SITE 22: Xaa represents any amino acid residue
  SITE 23: Xaa represents any amino acid residue
  SITE 26: Xaa represents any amino acid residue
  SITE 28: Xaa represents any amino acid residue
  SITE 29: Xaa represents any amino acid residue
  SITE 31: Xaa represents any amino acid residue
  SITE 33: Xaa represents any amino acid residue
  SITE 34: Xaa represents any amino acid residue
  SITE 38: Xaa represents any amino acid residue
  SITE 39: Xaa represents any amino acid residue
PXXPXXXXPXXPXSXYXXLXPXXPEXYXXLXPXXPDYXXL

SEQ ID NO: 28 - protein, 282 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 56: Xaa represents any amino acid residue
  SITE 57: Xaa represents any amino acid residue
  SITE 59: Xaa represents any amino acid residue
  SITE 60..108: Xaa represents any amino acid residue or is absent
  SITE 110: Xaa represents any amino acid residue
  SITE 112: Xaa represents any amino acid residue
  SITE 113: Xaa represents any amino acid residue
  SITE 115: Xaa represents any amino acid residue
  SITE 116..164: Xaa represents any amino acid residue or is absent
  SITE 166: Xaa represents any amino acid residue
  SITE 167: Xaa represents any amino acid residue
  SITE 170: Xaa represents any amino acid residue
  SITE 171..219: Xaa represents any amino acid residue or is absent
  SITE 221: Xaa represents any amino acid residue
  SITE 222: Xaa represents any amino acid residue
  SITE 224: Xaa represents any amino acid residue
  SITE 225..273: Xaa represents any amino acid residue or is absent
  SITE 275: Xaa represents any amino acid residue
  SITE 276: Xaa represents any amino acid residue
  SITE 280: Xaa represents any amino acid residue
  SITE 281: Xaa represents any amino acid residue
PXXPXXXXXXXXXXXXXXXXXXXXXXXXXXXXXXXXXXXXXXXXXXXXXXXXXXPXXPXXXXXXXXXXXXXXXXXXXXXXXXXXXXXXXXXXXXXXXXXXXXXXXXXXSXYXXLXXXXXXXXXXXXXXXXXXXXXXXXXXXXXXXXXXXXXXXXXXXXXXXXXXPXXPEXXXXXXXXXXXXXXXXXXXXXXXXXXXXXXXXXXXXXXXXXXXXXXXXXXYXXLXXXXXXXXXXXXXXXXXXXXXXXXXXXXXXXXXXXXXXXXXXXXXXXXXXPXXPDYXXL

SEQ ID NO: 29 - protein, 44 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 7: Xaa represents any amino acid residue
  SITE 8: Xaa represents any amino acid residue
  SITE 10: Xaa represents any amino acid residue
  SITE 11: Xaa represents any amino acid residue
  SITE 13: Xaa represents any amino acid residue
  SITE 15: Xaa represents any amino acid residue
  SITE 17: Xaa represents any amino acid residue
  SITE 18: Xaa represents any amino acid residue
  SITE 20: Xaa represents any amino acid residue
  SITE 22: Xaa represents any amino acid residue
  SITE 23: Xaa represents any amino acid residue
  SITE 26: Xaa represents any amino acid residue
  SITE 28: Xaa represents any amino acid residue
  SITE 29: Xaa represents any amino acid residue
  SITE 31: Xaa represents any amino acid residue
  SITE 33: Xaa represents any amino acid residue
  SITE 34: Xaa represents any amino acid residue
  SITE 38: Xaa represents any amino acid residue
  SITE 39: Xaa represents any amino acid residue
  SITE 41: Xaa represents any amino acid residue
  SITE 42: Xaa represents any amino acid residue
  SITE 43: Xaa represents any amino acid residue
  SITE 44: Xaa represents any amino acid residue
PXXPXXXXPXXPXSXYXXLXPXXPEXYXXLXPXXPDYXXLXXXX

SEQ ID NO: 30 - protein, 286 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 56: Xaa represents any amino acid residue
  SITE 57: Xaa represents any amino acid residue
  SITE 59: Xaa represents any amino acid residue
  SITE 60..108: Xaa represents any amino acid residue or is absent
  SITE 110: Xaa represents any amino acid residue
  SITE 112: Xaa represents any amino acid residue
  SITE 113: Xaa represents any amino acid residue
  SITE 115: Xaa represents any amino acid residue
  SITE 116..164: Xaa represents any amino acid residue or is absent
  SITE 166: Xaa represents any amino acid residue
  SITE 167: Xaa represents any amino acid residue
  SITE 170: Xaa represents any amino acid residue
  SITE 171..219: Xaa represents any amino acid residue or is absent
  SITE 221: Xaa represents any amino acid residue
  SITE 222: Xaa represents any amino acid residue
  SITE 224: Xaa represents any amino acid residue
  SITE 225..273: Xaa represents any amino acid residue or is absent
  SITE 275: Xaa represents any amino acid residue
  SITE 276: Xaa represents any amino acid residue
  SITE 280: Xaa represents any amino acid residue
  SITE 281: Xaa represents any amino acid residue
  SITE 283: Xaa represents any amino acid residue
  SITE 284: Xaa represents any amino acid residue
  SITE 285: Xaa represents any amino acid residue
  SITE 286: Xaa represents any amino acid residue
PXXPXXXXXXXXXXXXXXXXXXXXXXXXXXXXXXXXXXXXXXXXXXXXXXXXXXPXXPXXXXXXXXXXXXXXXXXXXXXXXXXXXXXXXXXXXXXXXXXXXXXXXXXXSXYXXLXXXXXXXXXXXXXXXXXXXXXXXXXXXXXXXXXXXXXXXXXXXXXXXXXXPXXPEXXXXXXXXXXXXXXXXXXXXXXXXXXXXXXXXXXXXXXXXXXXXXXXXXXYXXLXXXXXXXXXXXXXXXXXXXXXXXXXXXXXXXXXXXXXXXXXXXXXXXXXXPXXPDYXXLXXXX

SEQ ID NO: 31 - protein, 20 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 7: Xaa represents any amino acid residue
  SITE 8: Xaa represents any amino acid residue
  SITE 9: Xaa represents any amino acid residue
  SITE 10: Xaa represents any amino acid residue
  SITE 11: Xaa represents any amino acid residue
  SITE 12: Xaa represents any amino acid residue
  SITE 13: Xaa represents any amino acid residue
  SITE 14: Xaa represents any amino acid residue
  SITE 15: Xaa represents any amino acid residue
  SITE 16: Xaa represents any amino acid residue
  SITE 18: Xaa represents any amino acid residue
  SITE 19: Xaa represents any amino acid residue
PXXPXXXXXXXXXXXXYXXL

SEQ ID NO: 32 - protein, 20 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 7: Xaa represents any amino acid residue
  SITE 8: Xaa represents any amino acid residue
  SITE 9: Xaa represents any amino acid residue
  SITE 10: Xaa represents any amino acid residue
  SITE 11: Xaa represents any amino acid residue
  SITE 12: Xaa represents any amino acid residue
  SITE 13: Xaa represents any amino acid residue
  SITE 14: Xaa represents any amino acid residue
  SITE 15: Xaa represents any amino acid residue
  SITE 18: Xaa represents any amino acid residue
  SITE 19: Xaa represents any amino acid residue
PXXPXXXXXXXXXXXDYXXL

SEQ ID NO: 33 - protein, 20 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 8: Xaa represents any amino acid residue
  SITE 9: Xaa represents any amino acid residue
  SITE 10: Xaa represents any amino acid residue
  SITE 11: Xaa represents any amino acid residue
  SITE 12: Xaa represents any amino acid residue
  SITE 13: Xaa represents any amino acid residue
  SITE 14: Xaa represents any amino acid residue
  SITE 15: Xaa represents any amino acid residue
  SITE 16: Xaa represents any amino acid residue
  SITE 18: Xaa represents any amino acid residue
  SITE 19: Xaa represents any amino acid residue
PXXPXXYXXXXXXXXXYXXL

SEQ ID NO: 34 - protein, 20 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 8: Xaa represents any amino acid residue
  SITE 9: Xaa represents any amino acid residue
  SITE 10: Xaa represents any amino acid residue
  SITE 11: Xaa represents any amino acid residue
  SITE 12: Xaa represents any amino acid residue
  SITE 13: Xaa represents any amino acid residue
  SITE 14: Xaa represents any amino acid residue
  SITE 15: Xaa represents any amino acid residue
  SITE 18: Xaa represents any amino acid residue
  SITE 19: Xaa represents any amino acid residue
PXXPXXYXXXXXXXXDYXXL

SEQ ID NO: 35 - protein, 20 aa, Artificial Sequence
  Pilot peptide
  SITE 2: Xaa represents any amino acid residue
  SITE 3: Xaa represents any amino acid residue
  SITE 6: Xaa represents any amino acid residue
  SITE 8: Xaa represents any amino acid residue
  SITE 9: Xaa represents any amino acid residue
  SITE 11: Xaa represents any amino acid residue
  SITE 13: Xaa represents any amino acid residue
  SITE 14: Xaa represents any amino acid residue
  SITE 18: Xaa represents any amino acid residue
  SITE 19: Xaa represents any amino acid residue
PXXPEXYXXLXPXXPDYXXL

SEQ ID NO: 36 - protein, 58 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 56: Xaa represents any amino acid residue
  SITE 57: Xaa represents any amino acid residue
PXXPXXXXXXXXXXXXXXXXXXXXXXXXXXXXXXXXXXXXXXXXXXXXXXXXXXYXXL

SEQ ID NO: 37 - protein, 59 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 57: Xaa represents any amino acid residue
  SITE 58: Xaa represents any amino acid residue
PXXPXXXXXXXXXXXXXXXXXXXXXXXXXXXXXXXXXXXXXXXXXXXXXXXXXXDYXXL

SEQ ID NO: 38 - protein, 109 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 56: Xaa represents any amino acid residue
  SITE 57..105: Xaa represents any amino acid residue or is absent
  SITE 107: Xaa represents any amino acid residue
  SITE 108: Xaa represents any amino acid residue
PXXPXXXXXXXXXXXXXXXXXXXXXXXXXXXXXXXXXXXXXXXXXXXXXXXXXXYXXXXXXXXXXXXXXXXXXXXXXXXXXXXXXXXXXXXXXXXXXXXXXXXXXYXXL

SEQ ID NO: 39 - protein, 110 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 56: Xaa represents any amino acid residue
  SITE 57..105: Xaa represents any amino acid residue or is absent
  SITE 108: Xaa represents any amino acid residue
  SITE 109: Xaa represents any amino acid residue
PXXPXXXXXXXXXXXXXXXXXXXXXXXXXXXXXXXXXXXXXXXXXXXXXXXXXXYXXXXXXXXXXXXXXXXXXXXXXXXXXXXXXXXXXXXXXXXXXXXXXXXXXDYXXL

SEQ ID NO: 40 - protein, 118 aa, Artificial Sequence
  Pilot peptide
  SITE 2: Xaa represents any amino acid residue
  SITE 3: Xaa represents any amino acid residue
  SITE 6: Xaa represents any amino acid residue
  SITE 7..55: Xaa represents any amino acid residue or is absent
  SITE 57: Xaa represents any amino acid residue
  SITE 58: Xaa represents any amino acid residue
  SITE 60: Xaa represents any amino acid residue
  SITE 61..109: Xaa represents any amino acid residue or is absent
  SITE 111: Xaa represents any amino acid residue
  SITE 112: Xaa represents any amino acid residue
  SITE 116: Xaa represents any amino acid residue
  SITE 117: Xaa represents any amino acid residue
PXXPEXXXXXXXXXXXXXXXXXXXXXXXXXXXXXXXXXXXXXXXXXXXXXXXXXXYXXLXXXXXXXXXXXXXXXXXXXXXXXXXXXXXXXXXXXXXXXXXXXXXXXXXXPXXPDYXXL

SEQ ID NO: 41 - protein, 40 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 7: Xaa represents any amino acid residue
  SITE 8: Xaa represents any amino acid residue
  SITE 10: Xaa represents any amino acid residue
  SITE 11: Xaa represents any amino acid residue
  SITE 13: Xaa represents any amino acid residue
  SITE 14: Xaa represents any amino acid residue
  SITE 15: Xaa represents any amino acid residue
  SITE 17: Xaa represents any amino acid residue
  SITE 18: Xaa represents any amino acid residue
  SITE 20: Xaa represents any amino acid residue
  SITE 22: Xaa represents any amino acid residue
  SITE 23: Xaa represents any amino acid residue
  SITE 26: Xaa represents any amino acid residue
  SITE 28: Xaa represents any amino acid residue
  SITE 29: Xaa represents any amino acid residue
  SITE 31: Xaa represents any amino acid residue
  SITE 33: Xaa represents any amino acid residue
  SITE 34: Xaa represents any amino acid residue
  SITE 38: Xaa represents any amino acid residue
  SITE 39: Xaa represents any amino acid residue
PXXPXXXXPXXPXXXYXXLXPXXPEXYXXLXPXXPDYXXL

SEQ ID NO: 42 - protein, 280 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 56: Xaa represents any amino acid residue
  SITE 57: Xaa represents any amino acid residue
  SITE 59: Xaa represents any amino acid residue
  SITE 60..108: Xaa represents any amino acid residue or is absent
  SITE 110: Xaa represents any amino acid residue
  SITE 111: Xaa represents any amino acid residue
  SITE 113: Xaa represents any amino acid residue
  SITE 114..162: Xaa represents any amino acid residue or is absent
  SITE 164: Xaa represents any amino acid residue
  SITE 165: Xaa represents any amino acid residue
  SITE 168: Xaa represents any amino acid residue
  SITE 169..217: Xaa represents any amino acid residue or is absent
  SITE 219: Xaa represents any amino acid residue
  SITE 220: Xaa represents any amino acid residue
  SITE 222: Xaa represents any amino acid residue
  SITE 223..271: Xaa represents any amino acid residue or is absent
  SITE 273: Xaa represents any amino acid residue
  SITE 274: Xaa represents any amino acid residue
  SITE 278: Xaa represents any amino acid residue
  SITE 279: Xaa represents any amino acid residue
PXXPXXXXXXXXXXXXXXXXXXXXXXXXXXXXXXXXXXXXXXXXXXXXXXXXXXPXXPXXXXXXXXXXXXXXXXXXXXXXXXXXXXXXXXXXXXXXXXXXXXXXXXXXYXXLXXXXXXXXXXXXXXXXXXXXXXXXXXXXXXXXXXXXXXXXXXXXXXXXXXPXXPEXXXXXXXXXXXXXXXXXXXXXXXXXXXXXXXXXXXXXXXXXXXXXXXXXXYXXLXXXXXXXXXXXXXXXXXXXXXXXXXXXXXXXXXXXXXXXXXXXXXXXXXXPXXPDYXXL

SEQ ID NO: 43 - protein, 44 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6: Xaa represents any amino acid residue
  SITE 7: Xaa represents any amino acid residue
  SITE 8: Xaa represents any amino acid residue
  SITE 10: Xaa represents any amino acid residue
  SITE 11: Xaa represents any amino acid residue
  SITE 13: Xaa represents any amino acid residue
  SITE 14: Xaa represents any amino acid residue
  SITE 15: Xaa represents any amino acid residue
  SITE 17: Xaa represents any amino acid residue
  SITE 18: Xaa represents any amino acid residue
  SITE 20: Xaa represents any amino acid residue
  SITE 22: Xaa represents any amino acid residue
  SITE 23: Xaa represents any amino acid residue
  SITE 26: Xaa represents any amino acid residue
  SITE 28: Xaa represents any amino acid residue
  SITE 29: Xaa represents any amino acid residue
  SITE 31: Xaa represents any amino acid residue
  SITE 33: Xaa represents any amino acid residue
  SITE 34: Xaa represents any amino acid residue
  SITE 38: Xaa represents any amino acid residue
  SITE 39: Xaa represents any amino acid residue
  SITE 41: Xaa represents any amino acid residue
  SITE 42: Xaa represents any amino acid residue
  SITE 43: Xaa represents any amino acid residue
  SITE 44: Xaa represents any amino acid residue
PXXPXXXXPXXPXXXYXXLXPXXPEXYXXLXPXXPDYXXLXXXX

SEQ ID NO: 44 - protein, 284 aa, Artificial Sequence
  Pilot peptide
  SITE 2: Xaa represents any amino acid residue
  SITE 3: Xaa represents any amino acid residue
  SITE 5: Xaa represents any amino acid residue
  SITE 6..54: Xaa represents any amino acid residue or is absent
  SITE 56: Xaa represents any amino acid residue
  SITE 57: Xaa represents any amino acid residue
  SITE 59: Xaa represents any amino acid residue
  SITE 60..108: Xaa represents any amino acid residue or is absent
  SITE 110: Xaa represents any amino acid residue
  SITE 111: Xaa represents any amino acid residue
  SITE 113: Xaa represents any amino acid residue
  SITE 114..162: Xaa represents any amino acid residue or is absent
  SITE 164: Xaa represents any amino acid residue
  SITE 165: Xaa represents any amino acid residue
  SITE 168: Xaa represents any amino acid residue
  SITE 169..217: Xaa represents any amino acid residue or is absent
  SITE 219: Xaa represents any amino acid residue
  SITE 220: Xaa represents any amino acid residue
  SITE 222: Xaa represents any amino acid residue
  SITE 223..271: Xaa represents any amino acid residue or is absent
  SITE 273: Xaa represents any amino acid residue
  SITE 274: Xaa represents any amino acid residue
  SITE 278: Xaa represents any amino acid residue
  SITE 279: Xaa represents any amino acid residue
  SITE 281: Xaa represents any amino acid residue
  SITE 282: Xaa represents any amino acid residue
  SITE 283: Xaa represents any amino acid residue
  SITE 284: Xaa represents any amino acid residue
PXXPXXXXXXXXXXXXXXXXXXXXXXXXXXXXXXXXXXXXXXXXXXXXXXXXXXPXXPXXXXXXXXXXXXXXXXXXXXXXXXXXXXXXXXXXXXXXXXXXXXXXXXXXYXXLXXXXXXXXXXXXXXXXXXXXXXXXXXXXXXXXXXXXXXXXXXXXXXXXXXPXXPEXXXXXXXXXXXXXXXXXXXXXXXXXXXXXXXXXXXXXXXXXXXXXXXXXXYXXLXXXXXXXXXXXXXXXXXXXXXXXXXXXXXXXXXXXXXXXXXXXXXXXXXXPXXPDYXXLXXXX

SEQ ID NO: 45 - protein, 27 aa, Artificial Sequence
  T4-fibritin ???Foldon??? trimerization domain
GYIPEAPRDGQAYVRKDGEWVLLSTFL

SEQ ID NO: 46 - protein, 5 aa, Artificial Sequence
  Peptide targeting the MPER subdomain
PWYIW

SEQ ID NO: 47 - protein, 6 aa, Artificial Sequence
  Peptide targeting the MPER subdomain
WPWYIW

SEQ ID NO: 48 - protein, 7 aa, Artificial Sequence
  Peptide targeting the MPER subdomain
KWPWYIW

SEQ ID NO: 49 - protein, 8 aa, Artificial Sequence
  Peptide targeting the MPER subdomain
IKWPWYIW

SEQ ID NO: 50 - protein, 9 aa, Artificial Sequence
  Peptide targeting the MPER subdomain
YIKWPWYIW

SEQ ID NO: 51 - protein, 10 aa, Artificial Sequence
  Peptide targeting the MPER subdomain
QYIKWPWYIW

SEQ ID NO: 52 - protein, 11 aa, Artificial Sequence
  Peptide targeting the MPER subdomain
EQYIKWPWYIW

SEQ ID NO: 53 - protein, 12 aa, Artificial Sequence
  Peptide targeting the MPER subdomain
YEQYIKWPWYIW

SEQ ID NO: 54 - protein, 13 aa, Artificial Sequence
  Peptide targeting the MPER subdomain
KYEQYIKWPWYIW

SEQ ID NO: 55 - protein, 14 aa, Artificial Sequence
  Peptide targeting the MPER subdomain
GKYEQYIKWPWYIW

SEQ ID NO: 56 - protein, 15 aa, Artificial Sequence
  Peptide targeting the MPER subdomain
LGKYEQYIKWPWYIW